(19) United States
(12) Patent Application Publication
(10) Pub. No.: US 2021/0228130 A1
(43) Pub. Date: Jul. 29, 2021

(54) METHOD, DEVICE AND SYSTEM FOR ASSESSING AN AUTISM SPECTRUM DISORDER

(71) Applicant: Hoffmann-La Roche Inc., Little Falls, NJ (US)

(72) Inventors: Christian Gossens, Basel (CH); Timothy Kilchenmann, Basel (CH); Joerg Hipp, Basel (CH); David Nobbs, Basel (CH); Christopher Chatham, Basel (CH); Michael Lindemann, Basel (CH)

(21) Appl. No.: 17/221,567

(22) Filed: Apr. 2, 2021

Related U.S. Application Data
(63) Continuation of application No. PCT/EP2019/076975, filed on Oct. 4, 2019.

(30) Foreign Application Priority Data
Oct. 5, 2018 (EP) 18 198 954.2
May 3, 2019 (EP) 19 172 539.9

(51) Int. Cl. A61B 5/16; A61B 5/00; A61B 5/024; A61B 5/11

(52) U.S. Cl. CPC A61B 5/168; A61B 5/4842; A61B 5/6898; G16H 50/70; A61B 5/02405; A61B 5/4839; A61B 5/1118; A61B 5/0022

(57) ABSTRACT

The present disclosure relates to the field of schizophrenia or an autism spectrum disorder (“ASD”) diagnostics and disease management. Specifically, the present disclosure teaches a method of assessing schizophrenia or ASD in a subject in which a subject's usage data for a mobile device is collected over a first predefined time window. A usage behavior parameter is determined from the usage data, and the determined usage behavior parameter is compared to a reference. From the comparison it may be determined whether the schizophrenia or ASD in the subject is improving, persisting or worsening. A system including a mobile device having sensors recording usage data and a remote device operatively linked to the mobile device is also disclosed.

### RELATED APPLICATIONS

This application is a continuation of PCT/EP2019/076975, filed Oct. 4, 2019, which claims priority to EP 18198954.2, filed Oct. 5, 2018, and EP 19172539.9 filed May 3, 2019, the entire disclosures of both of which are hereby incorporated herein by reference.

### BACKGROUND

The present disclosure relates to the field of schizophrenia or an autism spectrum disorder diagnostics and disease management. Specifically, it relates to a method of assessing schizophrenia or an autism spectrum disorder in a subject comprising the steps of determining at least one usage behavior parameter from a dataset comprising usage data for a mobile device within a first predefined time window wherein said mobile device has been used by the subject and comparing the determined at least one usage behavior parameter to a reference, whereby schizophrenia or an autism spectrum disorder will be assessed. The present disclosure also relates to a mobile device comprising a processor, at least one sensor recording usage data and a database as well as software which is tangibly embedded to said device and, when running on said device, carries out the aforementioned method. Also contemplated by the disclosure is a system comprising a mobile device comprising at least one sensor recording usage data and a remote device comprising a processor and a database as well as software, which is tangibly embedded to said device and, when running on said device, carries out the aforementioned method, wherein said mobile device and said remote device are operatively linked to each other. Also, the disclosure relates to the use of the mobile device or the system for assessing schizophrenia by analyzing a dataset comprising usage data for a mobile device within a first predefined time window wherein said mobile device has been used by the subject.

Autism spectrum disorders are neurodevelopmental disorders including classical autism and related medical conditions. Autism spectrum disorders appear to have a prevalence of about 1 of 59 (Surveillance Summaries/Apr. 27, 2018/67(6); 1-23). The rates appear to be consistent among different cultural and ethnic backgrounds. However, males appear to be affected more often than females.

Typical symptoms include problems in social communication and social interaction, and restricted, repetitive patterns of behavior, interests or activities. Symptoms are usually recognized between 2 and 4 years of age. Long-term issues may include difficulties in creating and keeping relationships, maintaining a job, and performing daily tasks.

The DSM 5 recognizes autism, Asperger syndrome, pervasive developmental disorder not otherwise specified (PDD-NOS), and childhood disintegrative disorder as disorders falling into the group of autism spectrum disorders. Common comorbid symptoms include anxiety and sleep problems. Genetic reasons as well as environmental influences are discussed as potential risk factors.

Various diagnostic tests and behavior characteristics have been described already for assessing ASD. For example, individuals with ASD can have unusual vocal properties, a reduced amount of speech and difficulty with turn-taking (Capps et al., 1998). They may intensely focus on their restricted interest making conversations difficult (Rouhizadeh, 2015). 66% of individuals with ASD have a history of aggressive episodes (Kanne and Micah, 2011). Individuals with ASD are less likely to engage in social approaches, to interact with others (Corbett et al., 2010) and to have a strict adherence to routines (Henderson et al., 2011). Recent studies have also demonstrated the potential for automated detection of repetitive movements (GroBekathofer et al., 2017).

Individuals with ASD have significant problems with sleep including prolonged sleep latency, decreased sleep efficiency, reduced total sleep time, increased waking after sleep-onset and daytime sleepiness (Cohen et al., 2014). Poor sleepers with ASD have greater affective problems and poorer social interactions (Malow et al., 2006). The co-morbidity of anxiety disorders with ASD is estimated to be 39.6% (Steensel et al., 2011). Anxiety is associated with lower rates of heart-rate variability (Friedman, 2007). The Reading the Mind in the Eyes Test (RMET) is a well established assessment of the ability to recognize the mental states of others, developed by Baron Cohen et al. (2001), in ASD.

Individuals with autism can have difficulty with working memory. They are more likely to make errors than non-ASD individuals on the CANTAB assessment of spatial working memory, and are less likely to consistently use a specific organized search strategy (Steele et al., 2007). “Stag Hunt” (named Treasure Hunt for this app) was developed to assess the cooperative ability of individuals with ASD. Difficulty in representing the strategy of another player has been shown to predict symptom severity (Yoshida et al., 2010). People with ASD show distinctive, atypical acoustic patterns of speech (Fusaroli et al., 2017) and a tendency to fixate on non-social elements of images, such as those used in the ADOS (Mouga et al., 2015).

Some domains of ASD referred to above are affected in other diseases as well such as Angelman Syndrome, Schizophrenia and multiple sclerosis (MS). Social cognition focuses on how people process, store, and apply information about other people and social situations, thus guiding their social interactions. Recent evidence has shown 20% of social cognitive impairment among patients with MS and 20-40% of social cognition impairment in MS patients are in the theory of the mind tasks, as well as in the social perception tasks to recognize certain negative facial emotion expressions (Dulau C et al 2017, Journal of Neurology, 264 (4): 740-748). Theory of the mind defined as one's ability to represent the psychological perspective of interacting subjects, requiring an internal theorization about their thoughts and beliefs, emotions, affective states, and feelings are highly affected in the MS population. Emotion recognition is a part of social perception, defined as one's ability to perceive information about the mental state of other subjects based on behavioral signals, also known to be affected in MS.

Certain pharmaceuticals and pathways are known in the treatment of ASD, such as Balovaptan (Sci Transl Med. 2019 May 8; 11(491)), Arbaclofen (J Autism Dev Disord. 2014 April; 44(4):958-64) GABA A (Volume 86, Issue 5, 3 Jun. 2015, Pages 1119-1130, RG7816 (https://adisinsight.springer.com/drugs/800051347), CM-AT (https://psych.ucsf.edu/CM-AT-autism), mGlu4/7 PAM (Front Mol Neurosci. 2018; 11: 387, Neuropsychopharmacology. 2014 August; 39(9): 2049-2060), Bumetanide (Ann Pharmacother. 2019 May; 53(5):537-544), JNJ-5279 (https://adisinsight.springer.com/drugs/800036911), L1-79 (Clin Ther. 2019 Sep. 3. pii: S0149-2918(19)30396-0), Tideglusib (https://www.thepharmaletter.com/article/positive-data-for-amo-02-in-autism-spectrum-disorder), FSM® (https://www.healio.com/pediatrics/autism-spectrum-disorders/news/online/%7B6b8a390d-1f6a-4f24-ac73-ee831f0c20e0%7D/fda-fast-tracks-microbiota-therapy-for-children-with-autism), donepezil (NCT01098383) AB-2004 (http://www.microbiometimes.com/axial-biotherapeutics-announces-publication-of-preclinical-data-highlighting-the-link-between-human-gut-microbiota-and-behavioral-symptoms-of-autism- spectrum-disorder-in-mouse-models/), Zygel (https://zynerba.com/zynerba-pharmaceuticals-initiates-phase-2-trial-of-zygel-in-autism-spectrum-disorder/), OPN-300 (https://adisinsight.springer.com/trials/700258780), Ziprasidone (NCT00208559) (https://www.clinicaltrialsarena.com/news/oryzon-vafidemstat-autism-results/), Lurasidone (EudraCT Number: 2013-001694-24), Cannabidivarin (Front Cell Neurosci. 2019 Aug. 9; 13:367), RVT-701(https://adisinsight.springer.com/drugs/800047745), naloxone and naltrexone (Am J Ment Retard. 1989 May; 93(6):644-51), Risperidone (J Child Adolesc Psychopharmacol. 2008 June; 18(3): 227-236), Fatty Acids Omega-3 Treatment (EudraCT Number: 2007-006444-21), folinic acid (EudraCT Number: 2015-000955-25), Fluoxetine (EudraCT Number: 2008-003712-36).

There is a need for reliable measures for assessing autism spectrum disorders in affected patients.

### SUMMARY

The technical problem underlying the present disclosure may be seen in the provision of means and methods complying with the aforementioned needs. The technical problem is addressed by the embodiments described herein below.

The present disclosure relates to a method assessing an autism spectrum disorder in a subject comprising the steps of:
a) determining at least one usage behavior parameter from a dataset comprising usage data for a mobile device within a first predefined time window wherein said mobile device has been used by the subject; and
b) comparing the determined at least one usage behavior parameter to a reference, whereby an autism spectrum disorder will be assessed.

Typically, the method further comprises the step of (c) determining an improvement, persistency or worsening of the negative symptoms associated with schizophrenia or autism spectrum disorders in a subject based on the comparison carried out in step (b).

In some embodiments, the method may also comprise prior to step (a) the step of obtaining from the subject using a mobile device a dataset of comprising usage data for a mobile device within a first predefined time window. However, typically the method is an ex vivo method carried out on an existing dataset comprising usage data for a mobile device within a first predefined time window which does not require any physical interaction with the said subject.

The present disclosure also relates to a method assessing an autism spectrum disorder (ASD) in a subject comprising the steps of:
a) determining at least one behavior parameter from a dataset comprising behavior data from a subject suffering from ASD from a first predefined time window; and
b) comparing the determined at least one behavior parameter to a reference, whereby ASD will be assessed.

The behavior data typically comprise one or more data selected from the group consisting of:
(i) data indicative for conversational skills and obsessive interest;
(ii) data indicative for sociability and routines;
(iii) data indicative for repetitive movements;
(iv) data indicative for sleep behavior;
(v) data indicative for anxiety;
(vi) data indicative for emotion recognition;
(vii) data indicative for spatial working memory;
(viii) data indicative for cooperation behavior; and
(ix) data indicative for image exploration capabilities, vocal properties and speaker recognition.

Typically, the method further comprises the step of (c) determining an improvement, persistency or worsening of the symptoms associated with ASD in a subject based on the comparison carried out in step (b).

In some embodiments, the method may also comprise prior to step (a) the step of obtaining from the subject using a mobile device a dataset of comprising behavior data for a mobile device within a first predefined time window. However, typically the method is an ex vivo method carried out on an existing dataset comprising behavior data for a mobile device within a first predefined time window which does not require any physical interaction with the said subject.

The method as referred to in accordance with the present disclosure includes a method which essentially consists of the aforementioned steps or a method which may include additional steps.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once, typically will be used only once when introducing the respective feature or element. In the following, in most cases, when referring to the respective feature or element, the expressions “at least one” or “one or more” will not be repeated, notwithstanding the fact that the respective feature or element may be present once or more than once.

Further, as used in the following, the terms “particularly”, “more particularly”, “specifically”, “more specifically”, “typically”, and “more typically” or similar terms are used in conjunction with additional/alternative features, without restricting alternative possibilities. Thus, features introduced by these terms are additional/alternative features and are not intended to restrict the scope of the claims in any way. The disclosure may, as the skilled person will recognize, be performed by using alternative features. Similarly, features introduced by “in an embodiment of the invention” or similar expressions are intended to be additional/alternative features, without any restriction regarding alternative embodiments of the invention, without any restrictions regarding the scope of the invention and without any restriction regarding the possibility of combining the features introduced in such way with other additional/alternative or non-additional/alternative features of the invention.

The method may be carried out on the mobile device by the subject once the dataset of comprising usage or behavior data for a mobile device within a first predefined time window has been acquired. In an embodiment, the terms “usage data” and “behavior data” for a mobile device are used interchangeably herein. Typically, the mobile device and the device acquiring the dataset may be physically identical, i.e., the same device. Such a mobile device shall have a data acquisition unit which typically comprises means for data acquisition, i.e., means which detect or measure either quantitatively or qualitatively physical parameters and transform them into electronic signals transmitted to the evaluation unit in the mobile device used for carrying out the method according to the disclosure. The data acquisition unit comprises means for data acquisition, i.e., means which detect or measure either quantitatively or qualitatively physical parameters and transform them into electronic signals transmitted to the device being remote from the mobile device and used for carrying out the method according to the disclosure. Typically, said means for data acquisition comprise at least one sensor. It will be understood that more than one sensor can be used in the mobile device, i.e., at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, pedometer, fingerprint detectors, touch sensors, voice recorders, light sensors, pressure sensors, location data detectors, cameras, GPS, and the like. The evaluation unit typically comprises a processor and a database as well as software that is tangibly embedded on said device and, when running on said device, carries out the method of the disclosure. More typically, such a mobile device may also comprise a user interface, such as a screen, which allows for providing the result of the analysis carried out by the evaluation unit to a user.

Alternatively, it may be carried out on a device being remote with respect to the mobile device that has been used to acquire the said dataset. In this case, the mobile device shall merely comprise means for data acquisition, i.e., means which detect or measure, either quantitatively or qualitatively, physical parameters and transform them into electronic signals transmitted to the device being remote from the mobile device and used for carrying out the method according to the disclosure. Typically, said means for data acquisition comprise at least one sensor. It will be understood that more than one sensor can be used in the mobile device, i.e., at least two, at least three, at least four, at least five, at least six, at least seven, at least eight, at least nine or at least ten or even more different sensors. Typical sensors used as means for data acquisition are sensors such as gyroscope, magnetometer, accelerometer, proximity sensors, thermometer, pedometer, fingerprint detectors, touch sensors, voice recorders, light sensors, pressure sensors, location data detectors, cameras, GPS, and the like. Thus, the mobile device and the device used for carrying out the method of the disclosure may be physically different devices. In this case, the mobile device may communicate with the device used for carrying out the method of the present disclosure by any means for data transmission. Such data transmission may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Accordingly, for carrying out the method of the present disclosure, the only requirement is the presence of a dataset comprising usage or behavior data for a mobile device within a first predefined time window obtained from a subject using a mobile device. The said dataset may also be transmitted or stored from the acquiring mobile device on a permanent or temporary memory device that subsequently can be used to transfer the data to the device used for carrying out the method of the present disclosure. The remote device which carries out the method of the disclosure in this setup typically comprises a processor and a database as well as software which is tangibly embedded on said device and, when running on said device, carries out the method of the disclosure. More typically, the said device may also comprise a user interface, such as a screen, which allows for providing the result of the analysis carried out by the evaluation unit to a user.

The term “assessing” as used herein refers to determining or providing an aid for diagnosing whether a subject suffers from ASD and/or exhibits one or more symptoms associated therewith. Typically, assessing as referred to herein comprises determining an improvement, persistency or worsening of said symptoms, more typically an improvement of the said symptoms. As will be understood by those skilled in the art, such an assessment, although preferred to be, may usually not be correct for 100% of the investigated subjects. The term, however, requires that a statistically significant portion of subjects can be correctly assessed. Whether a portion is statistically significant can be determined by the person skilled in the art using various well known statistical evaluation tools, e.g., determination of confidence intervals, p-value determination, Student's t-test, Mann-Whitney test, etc. Details may be found in Dowdy and Wearden, Statistics for Research, John Wiley & Sons, New York 1983. Typically envisaged confidence intervals are at least 50%, at least 60%, at least 70%, at least 80%, at least 90% or at least 95%. The p-values are, typically, 0.2, 0.1, 0.05. Thus, the method of the present disclosure, typically, aids the assessment of ASD by providing a means for evaluating a dataset comprising usage or behavior data within a first predefined time window. The term also encompasses any kind of diagnosing, monitoring or staging of ASD.

In an embodiment of the method of the disclosure, said assessing autism spectrum disorders (ASD) comprises assessing at least one symptom associated with ASD selected from the group consisting of: social communication and social interaction, and restricted, repetitive patterns of behavior, interests or activities. Typically, said assessing autism spectrum disorders comprises determining an improvement of the at least one symptom associated with autism spectrum disorders.

The term “autism spectrum disorder (ASD)” as used herein refers to a group of neurodevelopmental disorders including autism and related medical conditions. Typical symptoms include problems in social communication and social interaction, and restricted, repetitive patterns of behavior, interests or activities. In particular, the symptom may be difficulties in recognizing and/or interpreting non-verbal cues, difficulties in conversation, decreased speech and/or language capabilities, repetitive speech, obsessive and/or restricted interests, repetitive movements, excessive adherence to routines, withdrawn in social settings, disinterest in peers, sleep problems, short-term memory problems, and/or anxiety. Symptoms are usually recognized between one and two years of age. Long-term issues may include difficulties in creating and keeping relationships, maintaining a job, and performing daily tasks. The DSM 5 recognizes autism, Asperger syndrome, pervasive developmental disorder not otherwise specified (PDD-NOS), and childhood disintegrative disorder as disorders falling into the group of autism spectrum disorders. Genetic reasons as well as environmental influences are discussed as potential risk factors.

Drugs which may be used for treating ASD patients include neurotransmitter reuptake inhibitors (fluoxetine), tricyclic antidepressants (imipramine), anticonvulsants (lamotrigine), atypical antipsychotics (clozapine), and acetylcholinesterase inhibitors (rivastigmine).

The term “subject” as used herein, typically, relates to mammals. The subject in accordance with the present disclosure may, typically, suffer from or shall be suspected to suffer from ASD, i.e., it may already show some or all of the symptoms associated with the said disease.

In an embodiment of the method of the disclosure said subject is a human.

The term “at least one usage behavior parameter” means that one or more usage behavior parameter may be determined in accordance with the disclosure, i.e., at least two, at least three, at least four, at least five, at least six, at least seven, at least eight, at least nine or at least ten or even more different behavior parameters. Thus, there is no upper limit for the number of different usage behavior parameters which can be determined in accordance with the method of the present disclosure. Typically, however, there will be between one and twelve different usage behavior parameters determined per dataset of mobile device usage data. The term “usage behavior parameter” is, in an embodiment, used interchangeably with the term “behavior parameter”.

The term “usage behavior parameter” as used herein refers to a parameter which is indicative for the usage behavior of a subject, in an embodiment with respect to the mobile device. This typically includes the behavior of the subject more generally that is measured when the subject is wearing or carrying the device or being in physical proximity thereto. For example, the mobile device in accordance with the present disclosure may be a smartphone. The dataset to be applied in accordance with the present disclosure shall comprise usage data for said smartphone recorded over a predefined period of time. Based on said data, usage behavior parameters may be calculated which reflect the usage behavior of the subject with respect to the smartphone, e.g., the frequency, kind of usage or non-usage (passive usage) or usage intensity etc. More typically, the usage behavior parameter(s) shall be recorded variables selected from Table 1 and/or Table 2, below, in an embodiment are selected from the group consisting of: phone and app usage parameters, in particular, contacts (number of IDs), calls (frequency, time, duration, direction (i.e., incoming or outgoing calls)), messages SMS (frequency, number of characters used, duration, direction), application (App) usage (name of the App, frequency, time, duration), screen in use (frequency, time, duration), WIFI and/or bluetooth use (number of visible WIFI and/or bluetooth connections, number of used connections), ambient sound parameters, in particular, volume and pitch (volume power, time), speech classifier (frequency, time, duration), Mel-frequency cepstral coefficients, movement parameters, in particular, activity parameters (tri-axial acceleration (20 Hz), time), location (obfuscated GPS, i.e., distance and direction of travelling), and light and proximity parameters (amount of ambient light over time, proximity of objects over time). Moreover, the touch behavior parameters may be used as a behavior parameter(s) in accordance with the method of the present disclosure. Typically, touch interactions, in particular, touch down, swiping and touch up, length and directionality of the touch movement, Y-coordinate of the touch event only, time stamps, whether or not it occurred on the keyboard, and/or typing behavior, in particular, character type (letter, number, punctuation mark, editing characters, function key, emoji), actual character used only for the following character types: punctuation mark (e.g., full stops, exclamation marks, editing characters (e.g., space, delete, backspace), time stamps may be envisaged. More typically, the usage behavior parameter(s) shall, thus, be recorded variables selected from Table 4, below, in the case of an autism spectrum disorder.

In an embodiment, typical behavior parameters may be selected from the following:

Data Indicative for Conversational Skills and Obsessive Interest

These typically comprise data for voice characteristics, amount of speech and/or turn-taking behavior during conversations. More typically, a support person may record weekly a conversation with the subject to be investigated. From the recorded conversation, features are extracted that allow for spectral, semantic and sentiment analyses. Characteristics of the voice are identified such as pitch, volume, shimmer and jitter. Turn-taking behavior during conversation shall be analyzed as well as repeated reference to identical topics. Various computer-implemented speech analysis as well as deep learning algorithms can be used for the analysis.

Data Indicative for Sociability and Routines

These typically comprise data for social interaction and/or movement pattern. Time in social versus non-social rooms as well as time in proximity to other people can be determined by using bluetooth transmitters placed in the home and carried by household members. The rooms and household members can be labeled such that it is possible to analyze the location and interactions of the subject. Furthermore, entropy of location and body movements can be measured indicating irregularity or regularity of movement patterns of the subject during the day. This can be achieved by using a mobile device at the subject, such as a smart watch.

Data Indicative for Repetitive Movements

These typically comprise data for frequency and duration of repetitive and/or stereotype movements. The frequency and duration of repetitive movements performed by a subject can be measured by using a mobile device at the subject such as a smart watch. Repetitive movements can be identified by a support person and subsequent training of computer-implemented pattern recognition and deep learning algorithms on recorded and manually annotated movement data sets.

Data Indicative for Sleep Behavior

These typically comprise data for sleep latency, sleep efficiency, sleep time, waking after sleep onset and/or sleepiness. Sleep pattern can be extracted based on movement data over night for the subject. The subject, typically, wears a mobile device, such as a smart watch, over night at, e.g., two days per week. Time to sleep onset and/or sleep duration may be determined by a computer-implemented algorithm analyzing the sleep behavior data.

Data Indicative for Anxiety

These typically comprise data for heart rate variability. It has been known that anxiety is associated with reduced heart rate variability and is a comorbidity in ASD. Typically, heart rate variability, depending on social locations and unusual routines, is determined from datasets of heart rates continuously measured by mobile devices, such as a smart watch, at the subject. The mobile device records PPG signals from the subject and determines the location via GPS. Ambient noise recording may also be used for evaluating the social context of a social interaction.

Data Indicative for Emotion Recognition

These typically comprise data from a computer-implemented reading the mind in the eyes test (RMET), in particular, emotional intensity for recognizing emotions simulated by tasks in the test, response and decision time for performing tasks during the test. In the computer-implemented test on the mobile device, the subject is exposed to a static image of a facial expression. The intensity of emotion shown on the static image is varied by an adaptive algorithm. The subject must tap on the screen when it recognize the emotion on the image and label the degree or kind of emotion on a scale. More details on the test may be found in the accompanying Examples below.

Data Indicative for Spatial Working Memory

These typically comprise data from a computer-implemented test for working memory. The test, typically, can be implemented by a game as follows: A computer-implemented algorithm depicts a chicken on the screen of the mobile device. Said chicken lay eggs. The eggs become visualized if the subject taps on a chicken. A chicken can only lay an egg once. The subject must remember when the chicken depicted on the screen has laid an egg. A chicken can only be checked for eggs once. The algorithm records the number of trials where a subject checks a chicken twice or more and the number of trials where a subject checks a chicken that has laid already an egg. Based on these results, data are generated that are indicative for the working memory of a subject. More details on the test may be found in the accompanying Examples below.

Data Indicative for Cooperation Behavior

These typically comprise data from a computer-implemented test assessing cooperation behavior. In a variant, the behavior of the computer agent is changed in a controlled manner and the behavior of the subject is quantified according to whether the subject understands the intentions of the agent.

The test, typically, can be implemented by a game as follows: The subject plays a turn taking game against a computer agent. It may pursue a coin worth one times a currency or it may pursue treasure chest worth more than one times the currency if it cooperates with the other player which may be a computer-implemented algorithm or a real player which may or may not be known to the subject. The number of cooperation events is calculated and serves for generating data indicative for cooperation behavior. More details on the test may be found in the accompanying Examples below. In particular, the implemented test can be expanded in the following way: The subject observes two computers playing the game and has to place a bet on the outcome. This variant of the test has the potential to differentiate between the unwillingness to cooperate versus an inability to identify cooperation behavior.

Data Indicative for Image Exploration Capabilities, Vocal Properties and Speaker Recognition

These typically comprise data from a computer-implemented test for visually identifying social and non-social elements, voice characteristics, and/or speaker recognition by conversation and ambient sound. The subject is exposed to an image on the screen of the mobile device that contains social and non-social elements. It is asked to communicate and record what happens on the image. Finger motion tracking is used to investigate inspection time of the social and the non-social aspects of the image. Voice characteristics (pitch, volume, shimmer, jitter) are analyzed from the recordings. In addition, acoustic fingerprints are extracted for speaker identification in conversation and ambient sound data. Based on these recorded data, data indicative for image exploration capabilities, vocal properties and speaker recognition can be generated by computer-implemented pattern recognition and deep learning algorithms. It has been known that subjects suffering from ASD have a tendency to exhibit atypical acoustic patterns of speech and a tendency to focus on non-social elements of an image.

More typically, the at least one usage behavior parameter may be a combination of the aforementioned parameters. The following combinations may, e.g., be envisaged:
phone and app usage parameters, ambient sound, movement parameters, and light and proximity parameters;
phone and app usage parameters, movement parameters, and light and proximity parameters;
phone and app usage parameters, ambient sound, and light and proximity parameters;
phone and app usage parameters, ambient sound, and movement parameters;
ambient sound, movement parameters, and light and proximity parameters;
phone and app usage parameters and ambient sound;
phone and app usage parameters, and movement parameters;
phone and app usage parameters, and light and proximity parameters;
ambient sound, and movement parameters;
ambient sound, and light and proximity parameters.

In an embodiment, the at least one behavior parameter is any of the aforementioned combination in combination with a touch behavior parameter as set forth above.

In an embodiment of the method of the disclosure, said at least one usage behavior parameter is a recorded variable according to Table 1, 2 and/or 3, below.

More typically, the at least one behavior parameter may be a combination of the aforementioned parameters. The following combinations may, e.g., be envisaged:
(i) data indicative for conversational skills and obsessive interest;
(iv) data indicative for sleep behavior;
(v) data indicative for anxiety;
(vi) data indicative for emotion recognition;
(vii) data indicative for spatial working memory;
(viii) data indicative for cooperation behavior; and
(ix) data indicative for image exploration capabilities, vocal properties and speaker recognition
   or
(i) data indicative for conversational skills and obsessive interest;
(ii) data indicative for sociability and routines;
(iii) data indicative for repetitive movements;
(v) data indicative for anxiety;
(vi) data indicative for emotion recognition;
(vii) data indicative for spatial working memory;
(viii) data indicative for cooperation behavior; and
(ix) data indicative for image exploration capabilities, vocal properties and speaker recognition.

The term “dataset comprising usage data”, in an embodiment for a mobile device, refers to an entirety of data reflecting or indicating different uses or tasks carried out by or with the mobile device which have been recorded by or acquired from the mobile device within a first time window. The first time window as referred to in this context is a predefined time window wherein the subject uses or is suspected to use the mobile device, i.e., it is the time period during which the dataset is recorded or acquired. Usage data may be, typically, phone usage data, application (App) usage data, ambient noise data, movement capture data and/or location capture data. The first time window may be of any length which is suitable for recording data that can be used for deriving a meaningful at least one usage behavior parameter. For example, if the duration of a phone call will be measured, the said first time window will at least last over said phone call. Typically, the usage data are recorded over a standardized time window, e.g., one or more hour(s), one or more day(s), one or more week(s) or one or more month(s). Depending on the subject and the circumstances, the skilled artisan is well aware of how to select a suitable redefined first time window for the purpose of recording or acquiring a dataset comprising behavior data.

In an embodiment of the method of the disclosure, the said usage data for a mobile device comprise data selected from the group consisting of: phone usage data, application (App) usage data, ambient noise data, movement capture data and location capture data.

The term “mobile device” as used herein refers to any portable device (like mobile phone, smart watch and the like) which comprises at least one sensor and data-recording equipment suitable for obtaining the dataset comprising usage data, in an embodiment. This may also require a data processor and storage unit, voice recording devices, speakers as well as a display for receiving input from the subject on the mobile device. Moreover, from the activity of the subject, data shall be recorded and compiled to a dataset which is to be evaluated by the method of the present disclosure either on the mobile device itself or on a second device. Depending on the specific setup envisaged, it may be necessary that the mobile device comprises data transmission equipment in order to transfer the acquired dataset from the mobile device to a further device. Smartphones, portable multimedia devices or tablet computers are particularly well-suited as mobile devices according to the present disclosure. Alternatively, portable sensors with data recording and processing equipment may be used. However, mobile devices may, in an embodiment, also include speaker systems with data recorders such as the Echo or Alexa devices from Amazon or the Sonos system.

In an embodiment of the method of the disclosure, said mobile device is a smartphone, smartwatch, wearable sensor, portable multimedia device or tablet computer.

Determining at least one usage behavior parameter can be achieved either by directly deriving a desired measured value from the dataset comprising usage data within a first predefined time window wherein said mobile device has been used by the subject. Alternatively, the usage behavior parameter may integrate one or more measured values from the dataset and, thus, may be a derived from the dataset by mathematical operations such as calculations. Typically, the performance parameter is derived from the dataset by an automated algorithm, e.g., by a computer program which automatically derives the usage behavior parameter from the dataset when tangibly embedded on a data processing device fed by the said dataset.

The term “reference” as used herein refers to a discriminator which allows assessing the ASD and, preferably, an improvement of the symptoms associated therewith in a subject. Such a discriminator may be a value for the usage behavior parameter which is indicative for subjects suffering from ASD and, preferably, exhibiting the symptoms associated therewith or not suffering from ASD and, preferably, the symptoms associated therewith.

In principle, such a value for a reference may be derived from a subject or group of subjects known to suffer from ASD and, in particular, exhibiting the symptoms associated therewith. If the determined usage behavior parameter is identical to the reference or above a threshold derived from the reference, the subject can be identified as suffering from ASD and, preferably, the symptoms associated therewith. If the determined usage behavior parameter differs from the reference and, in particular, is below the said threshold, the subject shall be identified as not suffering from or having an improvement of ASD or at least having an improvement of the symptoms associated therewith.

Alternatively, the reference may be derived from a subject or group of subjects known not to suffer from ASD and, in particular, not exhibiting the symptoms associated therewith. If the determined performance parameter from the subject is identical to the reference or below a threshold derived from the reference, the subject can be identified as not suffering from ASD or at least having an improvement of the symptoms associated therewith. If the determined performance parameter differs from the reference and, in particular, is above the said threshold, the subject shall be identified as suffering from ASD and, preferably, the symptoms associated therewith.

More typically, the reference may be a previously determined usage behavior parameter from a comprising usage data for a mobile device within a second predefined time window wherein said mobile device has been used by the subject, wherein said second time window has been prior to the first time window. In such a case, a determined usage behavior parameter from the actual dataset that differs with respect to the previously determined usage behavior parameter shall be indicative for either an improvement or worsening depending on the previous status of the disease or a symptom accompanying it and the kind of usage represented by the usage behavior parameter. The skilled person knows based on the kind of usage and previous usage behavior parameter how the said parameter can be used as a reference. Typical differences between determined usage behavior parameters and references are reflected by the expected changes for the recorded variables being indicative for an improvement. These are listed in Table 1, 2 and/or 3, in an embodiment Table 4, below.

Typically, an improvement of at least one symptom associated with ASD is determined if the at least one usage behavior parameter improves compared to the reference as indicated in Table 1, 2 and/or 3, in an embodiment Table 4, below.

In an embodiment of the method of the disclosure, said reference is at least one usage behavior parameter which has been determined in a dataset comprising usage data within a second predefined time window prior to the first predefined time widow. The first and second time windows may be separated by a third predefined time period, i.e., a predefined monitoring period. Typically, such a period may also depend on the length of the first and second time windows and range from days to weeks to months to years depending on the disease progression, state or development or the duration of therapeutic measures for the individual subject.

Comparing the determined at least one usage behavior parameter to a reference can be achieved by an automated comparison algorithm implemented on a data processing device such as a computer. The values of a determined usage behavior parameter and a reference for said determined usage behavior parameter, as specified elsewhere herein in detail, are compared to each other. As a result of the comparison, it can be assessed whether the determined usage behavior parameter is identical or differs from or is in a certain relation to the reference (e.g., is larger or lower than the reference). Based on said assessment, the subject can be identified as suffering from ASD and, preferably, exhibiting the symptoms associated therewith (“rule-in”), or not (“rule-out”). For the assessment, the kind of reference will be taken into account as described elsewhere in connection with suitable references according to the disclosure.

Moreover, by determining the degree of difference between a determined usage behavior parameter and a reference, a quantitative assessment of ASD shall be possible. It is to be understood that an improvement, worsening or unchanged overall disease condition or of symptoms thereof can be determined by comparing an actually determined usage behavior parameter to an earlier determined one used as a reference. Based on quantitative differences in the value of the said usage behavior parameter, the improvement, worsening or unchanged condition can be determined and, optionally, also quantified. If other references, such as references from subjects with ASD are used, it will be understood that the quantitative differences are meaningful if a certain disease stage can be allocated to the reference collective. Relative to this disease stage, worsening, improvement or unchanged disease condition can be determined in such a case and, optionally, also quantified.

The assessment of ASD in the subject may be indicated to the subject or another person, such as a medical practitioner. Typically, this is achieved by displaying the assessment result on a display of the mobile device or the evaluation device. Alternatively, a recommendation for a therapy, such as a drug treatment, or for a certain life style, e.g., a certain nutritional diet or rehabilitation measures, is provided automatically to the subject or other person. To this end, the established diagnosis is compared to recommendations allocated to different diagnosis in a database. Once the established diagnosis matches one of the stored and allocated diagnoses, a suitable recommendation can be identified due to the allocation of the recommendation to the stored diagnosis matching the established diagnosis. Accordingly, it is typically envisaged that the recommendations and diagnoses are present in form of a relational database. However, other arrangements that allow for the identification of suitable recommendations are also possible and known to the skilled artisan.

Thus, the method of the present disclosure, in an embodiment, also encompasses determining whether an ASD therapy or a therapy for the symptoms associated therewith was successful, or not.

In such a case, typically, between the second and the first time window the subject has received an ASD therapy or a therapy for at least one of the symptoms associated therewith. More typically, said therapy is a drug-based therapy.

An improvement of at least one symptom associated with ASD is, typically, indicative for a successful therapy.

Moreover, the one or more usage behavior parameter may also be stored on the mobile device or indicated to the subject, typically, in real-time. The stored usage behavior parameter may be assembled into a time course or similar evaluation measures. Such evaluated performance parameters may be provided to the subject as a feedback for usage behavior investigated in accordance with the method of the disclosure. Typically, such a feedback can be provided in electronic format on a suitable display of the mobile device and can be linked to a recommendation for a therapy as specified above or rehabilitation measures.

Further, the evaluated usage behavior parameter may also be provided to medical practitioners in doctors' offices or hospitals as well as to other health care providers, such as, developers of diagnostic tests or drug developers in the context of clinical trials, health insurance providers or other stakeholders of the public or private health care system.

Typically, the method of the present disclosure for assessing ASD in a subject may be carried out as follows:

First, a usage behavior parameter is determined from an existing dataset, in an embodiment comprising usage data for a mobile device, of a first predefined time window wherein said mobile device has been used by the subject. Said dataset may have been transmitted from the mobile device to an evaluating device, such as a computer, or may be processed in the mobile device in order to derive the usage behavior parameter from the dataset.

Second, the determined usage behavior parameter is compared to a reference by, e.g., using a computer-implemented comparison algorithm carried out by the data processor of the mobile device or by the evaluating device, e.g., the computer. The result of the comparison is assessed with respect to the reference used in the comparison and based on the said assessment the subject will be identified as a subject suffering from ASD, or not, or exhibiting an improvement of the symptoms associated therewith, or not.

Third, the said result of the assessment is indicated to the subject or to another person, such as a medical practitioner. However, it will be understood that for a final clinical diagnosis or assessment further factors or parameters may be taken into account by the clinician.

Further, a recommendation for a therapy is provided automatically to the subject or another person. To this end, the established diagnosis is compared to recommendations allocated to different diagnoses in a database. Once the established diagnosis matches one of the stored and allocated diagnoses, a suitable recommendation can be identified due to the allocation of the recommendation to the stored diagnosis matching the established diagnosis. Typical recommendations involve therapy with neurotransmitter reuptake inhibitors (fluoxetine), tricyclic antidepressants (imipramine), anticonvulsants (lamotrigine), atypical antipsychotics (clozapine), and acetylcholinesterase inhibitors (rivastigmine). Moreover, psychological and/or social counselling are also suitable measures.

Moreover, the present disclosure also provides for a method for recommending a therapy for ASD comprising the steps of:
(a) assessing ASD by carrying out the method of the disclosure described before; and
(b) recommending a therapy for ASD based on the assessment provided in step (a).

The term “recommending”, as used herein, means establishing a proposal for a supportive measure or combinations thereof which could be applied to the subject. However, it is to be understood that applying the actual therapy may not be comprised by the term.

Typically, said therapy for ASD in this context comprises treatment by at least one drug selected from the group consisting of: a Vasopressin 1a antagonist, more particularly Balovaptan, a N-Methyl-D-Aspartate (NMDA) receptor antagonists, in particular memantine or RVT-701, a selective inhibitor of the enzyme fatty acid amide hydrolase (FAAH), in particular JNJ-5279, a GABA-modulator, in particular a GABA Aa5 positive allosteric modulator (PAM), in particular RG7816, a GABA A modulator or a selective GABA-B agonist, in particular arbaclofen, a mGlu4/7 positive allosteric modulator, oxytocin, in particular OPN-300, a Acetyl-Choline Esterase Inhibitor, in particular donepezil, a dual inhibitor of lysine (K)-specific demethylase 1A/monoamine oxidase B, in particular Vafidemstat, a tyrosine hydroxylase inhibitor, in particular L1-79, a selective and irreversible small molecule non-ATP-competitive glycogen synthase kinase 3 (GSK-3) inhibitor, in particular Tideglusib, an Amylase, lipase & protease regulator enzymes like CM-AT, a NKCC1 cation-chloride co-transporter blocker, in particular bumetamide, a microbiota transfer therapy, in particular FSM®, a microbiome modulator, in particular AB-2004, a selective serotonin reuptake inhibitor, in particular fluoxetine, a dopamine 2 receptor antagonist, in particular risperidone, ziprasidone or lurasidone, a non-euphoric cannabinoid, in particular Zygel, a phytocannabinoid, in particular Cannabidivarin, a mu-opioid receptor antagonist, in particular naloxone or naltrexone or Fatty Acids Omega-3 or folinic acid treatment.

As an alternative or in addition, the usage behavior parameter underlying the diagnosis will be stored on the mobile device. Typically, it shall be evaluated together with other stored performance parameters by suitable evaluation tools, such as time course assembling algorithms, implemented on the mobile device which can assist electronically with a therapy recommendation as specified elsewhere herein.

The disclosure, in light of the above, also specifically contemplates a method of assessing ASD and, preferably, an improvement of the symptoms associated therewith in a subject comprising the steps of:
a) obtaining from said subject using a mobile device a dataset comprising usage data from a mobile device within a first predefined time window wherein said mobile device has been used by the subject;
b) determining at least one usage behavior parameter determined from said dataset;
c) comparing the determined at least one usage behavior parameter to a reference; and
d) assessing ASD and, preferably, an improvement of the symptoms associated therewith in a subject based on the comparison carried out in step (b), typically, by determining whether the subject suffers from ASD or exhibits an improvement of the symptoms associated therewith, or not.

Advantageously, it has been found in the studies underlying the present disclosure that usage behavior parameters obtained from datasets comprising usage and other data for a mobile device within a first predefined time window wherein said mobile device has been used by the subject can be used to assess ASD in said subject. In particular, the said usage behavior parameters can be used to identify an improvement of the symptoms associated with ASD in said subject and, thus, aid monitoring of subjects, e.g., undergoing ASD therapy as specified elsewhere herein. The said datasets can be acquired from ASD patients in a convenient manner by using mobile devices such as the omnipresent smart phones, portable multimedia devices or tablet computers, in an embodiment sensor devices, on which the subjects perform active or passive tests, in an embodiment pressure tests. The datasets acquired can be subsequently evaluated by the method of the disclosure for the usage behavior parameter suitable as digital biomarker. Said evaluation can be carried out on the same mobile device or it can be carried out on a separate remote device. Moreover, by using such mobile devices, recommendations on therapeutic measures can be provided to the patients directly, i.e., without the consultation of a medical practitioner in a doctor's office or hospital or emergency medical provider. Thanks to the present disclosure, the life conditions of ASD patients can be adjusted more precisely to the actual disease status due to the use of an actual determined usage behavior parameter by the method of the disclosure. Thereby, drug treatments can be evaluated for efficacy and dosage regimens can be adapted to the current status of the patient. It is to be understood that the method of the disclosure is, typically, a data evaluation method which requires an existing dataset from a subject. Within this dataset, the method determines at least one usage behavior parameter which can be used for assessing ASD.

Accordingly, the method of the present disclosure may be used for:
assessing the disease condition;
monitoring patients, in particular, in a real life, daily situation and on large scale;
supporting patients with therapy recommendations;
investigating drug efficacy, e.g., also during clinical trials;
facilitating and/or aiding therapeutic decision making;
supporting hospital management;
supporting health insurance assessments and management; and/or
supporting decisions in public health management.

The present disclosure also contemplates a method for treating and/or preventing ASD in a subject suffering or suspect to suffer therefrom comprising
(a) assessing ASD by carrying out the method of the disclosure described before; and
(b) applying a therapy for ASD based on the assessment provided in step (a).

Typically, said therapy for ASD in this context comprises treatment by at least one drug selected from the group consisting: a Vasopressin 1a antagonist, more particularly Balovaptan, a N-Methyl-D-Aspartate (NMDA) receptor antagonists, in particular memantine or RVT-701, a selective inhibitor of the enzyme fatty acid amide hydrolase (FAAH), in particular JNJ-5279, a GABA-modulator, in particular a GABA Aa5 positive allosteric modulator (PAM), in particular RG7816, a GABA A modulator or a selective GABA-B agonist, in particular arbaclofen, a mGlu4/7 positive allosteric modulator, oxytocin, in particular OPN-300, a Acetyl-Choline Esterase Inhibitor, in particular donepezil, a dual inhibitor of lysine (K)-specific demethylase 1A/monoamine oxidase B, in particular Vafidemstat, a tyrosine hydroxylase inhibitor, in particular L1-79, a selective and irreversible small molecule non-ATP-competitive glycogen synthase kinase 3 (GSK-3) inhibitor, in particular Tideglusib, an Amylase, lipase & protease regulator enzymes like CM-AT, a NKCC1 cation-chloride co-transporter blocker, in particular bumetamide, a microbiota transfer therapy, in particular FSM®, a microbiome modulator, in particular AB-2004, a selective serotonin reuptake inhibitor, in particular fluoxetine, a dopamine 2 receptor antagonist, in particular risperidone, ziprasidone or lurasidone, a non-euphoric cannabinoid, in particular Zygel, a phytocannabinoid, in particular Cannabidivarin, a mu-opioid receptor antagonist, in particular naloxone or naltrexone or Fatty Acids Omega-3 or folinic acid treatment. Said drug is to be administered in the aforementioned method for treating and/or preventing ASD in a therapeutically effective amount to the subject. A therapeutically effective amount for treating or preventing ASD is known in the art for the aforementioned drugs and can be determined or adopted by the medical practitioner. Specifically, factors such as age, gender, weight, disease history, general health and well-being and the like, may be taken into account when determining a suitable dosage or dosage regimen.

The term “treating” as used herein, typically, refers to curing or ameliorating ASD or at least one of its symptoms. The term “preventing” as used herein, typically, refers to significantly reducing the likelihood of onset of ASD within a certain time window.

The present disclosure also contemplates a computer program, computer program product or computer readable storage medium having tangibly embedded said computer program, wherein the computer program comprises instructions that, when run on a data processing device or computer, carry out the method of the present disclosure as specified above. Specifically, the present disclosure further encompasses:
A computer or computer network comprising at least one processor, wherein the processor is adapted to perform the method according to one of the embodiments described,
a computer loadable data structure that is adapted to perform the method according to one of the embodiments described while the data structure is being executed on a computer,
a computer script, wherein the computer program is adapted to perform the method according to one of the embodiments described while the program is being executed on a computer,
a computer program comprising program means for performing the method according to one of the embodiments described while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described after having been loaded into a main and/or working storage of a computer or of a computer network,
a computer program product having program code means, wherein the program code means can be stored or are stored on a storage medium, for performing the method according to one of the embodiments if the program code means are executed on a computer or on a computer network,
a data stream signal, typically encrypted, comprising a dataset comprising usage data from a mobile device within a first predefined time window wherein said mobile device has been used by the subject, and
a data stream signal, typically encrypted, comprising the at least one usage behavior parameter derived from the dataset.

The present disclosure, further, relates to a method for determining at least one usage behavior parameter from a dataset comprising usage data from a mobile device within a first predefined time window wherein said mobile device has been used by the subject:
a) deriving at least one usage behavior parameter from said dataset; and
b) comparing the determined at least one usage behavior parameter to a reference, wherein, typically, said at least one usage behavior parameter can aid assessing ASD and, preferably, assessing an improvement of the symptoms associated therewith in said subject.

The present disclosure relates to a mobile device comprising a processor, at least one sensor recording usage behavior data and a database as well as software which is tangibly embedded to said device and, when running on said device, carries out the method of the disclosure.

The said mobile device is, thus, configured to be capable of acquiring the dataset and to determine the usage behavior parameter therefrom. Moreover, it is configured to carry out the comparison to a reference and to establish the assessment of ASD as described elsewhere herein in detail.

The present disclosure further relates to a system comprising a mobile device comprising at least one sensor recording usage data and a remote device comprising a processor and a database as well as software which is tangibly embedded to said device and, when running on said device, carries out the method of the disclosure, wherein said mobile device and said remote device are operatively linked to each other.

Under “operatively linked to each other” it is to be understood that the devices are connected so as to allow data transfer from one device to the other device. Typically, it is envisaged that at least the mobile device which acquires data from the subject is connected to the remote device carrying out the steps of the methods of the disclosure such that the acquired data can be transmitted for processing to the remote device. However, the remote device may also transmit data to the mobile device, such as signals controlling or supervising its proper function. The connection between the mobile device and the remote device may be achieved by a permanent or temporary physical connection, such as coaxial, fiber, fiber-optic or twisted-pair, 10 BASE-T cables. Alternatively, it may be achieved by a temporary or permanent wireless connection using, e.g., radio waves, such as Wi-Fi, LTE, LTE-advanced or Bluetooth. Further details may be found elsewhere in this specification. For data acquisition, the mobile device may comprise a user interface such as screen or other equipment for data acquisition.

The present disclosure further contemplates the use of the mobile device or the system of the disclosure for assessing ASD comprising analyzing a dataset comprising usage data from a mobile device within a first predefined time window wherein said mobile device has been used by the subject, typically, according to the method of the present disclosure.

In the following, further particular embodiments of the disclosure are listed:

### Embodiment 1

A method assessing schizophrenia or an autism spectrum disorder in a subject comprising the steps of:
a) determining at least one usage behavior parameter from a dataset comprising usage data for a mobile device within a first predefined time window wherein said mobile device has been used by the subject; and
b) comparing the determined at least one usage behavior parameter to a reference, whereby schizophrenia or an autism spectrum disorder will be assessed.

### Embodiment 2

The method of embodiment 1, wherein said assessing schizophrenia comprises assessing at least one negative symptom associated with schizophrenia selected from the group consisting of: asociality, alogia, apathy, anhedonia and impaired attention and wherein said assessing an autism spectrum disorder comprises assessing at least one negative symptom associated with an autism spectrum disorder selected from the group consisting of: social communication and social interaction, and restricted, repetitive patterns of behavior, interests or activities.

### Embodiment 3

The method of embodiment 2, wherein said assessing schizophrenia or an autism spectrum disorder comprises determining an improvement of the at least one negative symptom associated with schizophrenia or an autism spectrum disorder.

### Embodiment 4

The method of any one of embodiments 1 to 3, wherein the said usage data for a mobile device comprise data selected from the group consisting of: phone usage data, application (App) usage data, ambient noise data, movement capture data and location capture data.

### Embodiment 5

The method of any one of embodiments 1 to 4, wherein said at least one usage behavior parameter is a recorded variable according to Tables 1-4 in the case of an autism spectrum disorder.

### Embodiment 6

The method of embodiment 5, wherein an improvement of at least one negative symptom associated with schizophrenia or an autism spectrum disorder is determined if the at least one usage behavior parameter improves compared to the reference as indicated in Tables 1-4 in the case of an autism spectrum disorder.

### Embodiment 7

The method of any one of embodiments 1 to 6, wherein said reference is at least one usage behavior parameter which has been determined in a dataset comprising usage data for a mobile device within a second predefined time window prior to the first predefined time widow.

### Embodiment 8

The method of embodiment 7, wherein between the second and the first time window the subject has received a schizophrenia or an autism spectrum disorder therapy or a therapy for a negative symptom associated therewith.

### Embodiment 9

The method of embodiment 9, wherein said therapy is a drug-based therapy.

### Embodiment 10

The method of embodiment 8 or 9, wherein an improvement of at least one negative symptom associated with schizophrenia or an autism spectrum disorder is indicative for a successful therapy.

### Embodiment 11

The method of any one of embodiments 1 to 10, wherein said mobile device is a smartphone, smartwatch, wearable sensor, portable multimedia device or tablet computer.

### Embodiment 12

The method of any one of embodiments 1 to 11, wherein said subject is a human.

### Embodiment 13

A mobile device comprising a processor, at least one sensor recording usage data and a database as well as software which is tangibly embedded to said device and, when running on said device, carries out the method of any one of embodiments 1 to 12.

### Embodiment 14

A system comprising a mobile device comprising at least one sensor recording usage data and a remote device comprising a processor and a database as well as software which is tangibly embedded to said device and, when running on said device, carries out the method of any one of embodiments 1 to 12, wherein said mobile device and said remote device are operatively linked to each other.

### Embodiment 15

Use of the mobile device according to embodiment 13 or the system of embodiment 14 for assessing schizophrenia or an autism spectrum disorder by analyzing a dataset comprising usage data for a mobile device within a first predefined time window wherein said mobile device has been used by the subject.

### Embodiment 16

A method assessing an autism spectrum disorder (ASD) in a subject comprising the steps of:
a) determining at least one behavior parameter from a dataset comprising behavior data from a subject suffering from ASD from a first predefined time window; and
b) comparing the determined at least one behavior parameter to a reference, whereby ASD will be assessed, wherein said behavior data comprise one or more data selected from the group consisting of:
   (i) data indicative for conversational skills and obsessive interest;
   (ii) data indicative for sociability and routines;
   (iii) data indicative for repetitive movements;
   (iv) data indicative for sleep behavior;
   (v) data indicative for anxiety;
   (vi) data indicative for emotion recognition;
   (vii) data indicative for spatial working memory;
   (viii) data indicative for cooperation behavior; and
   (ix) data indicative for image exploration capabilities, vocal properties and speaker recognition.

### Embodiment 17

The method of embodiment 16, wherein said reference is at least one behavior parameter which has been determined in a dataset comprising behavior data within a second predefined time window prior to the first predefined time widow.

### Embodiment 18

The method of embodiment 16 or 17, wherein said assessing ASD comprises assessing at least one symptom associated with an autism spectrum disorder selected from the group consisting of: social communication and social interaction, and restricted, repetitive patterns of behavior, interests or activities.

### Embodiment 19

The method of embodiment 18, wherein said assessing ASD comprises determining an improvement of the at least one symptom associated with ASD.

### Embodiment 20

The method of embodiment 19, wherein an improvement of at least one symptom associated with ASD is determined if the at least one behavior parameter improves compared to the reference as indicated in Table 4.

### Embodiment 21

The method of any one of embodiments 16 to 20, wherein the said dataset comprising behavior data has been obtained from a mobile device.

### Embodiment 22

The method of any one of embodiments 16 to 21, wherein said data indicative for conversational skills and obsessive interest comprise data for voice characteristics, amount of speech and/or turn-taking behavior during conversations.

### Embodiment 23

The method of any one of embodiments 16 to 22, wherein said data indicative for sociability and routines comprise data for social interaction and/or movement pattern.

### Embodiment 24

The method of any one of embodiments 16 to 23, wherein said data indicative for data indicative for repetitive movements comprise data for frequency and duration of repetitive and/or stereotype movements.

### Embodiment 25

The method of any one of embodiments 16 to 24, wherein said data indicative for sleep behavior comprise data for sleep latency, sleep efficiency, sleep time, waking after sleep onset and/or sleepiness.

### Embodiment 26

The method of any one of embodiments 16 to 25, wherein said data indicative for anxiety comprise data for heart rate variability.

### Embodiment 27

The method of any one of embodiments 16 to 26, wherein said data indicative for emotion recognition comprise data from a computer-implemented reading the mind in the eyes test (RMET), in particular, emotional intensity for recognizing emotions simulated by tasks in the test, response and decision time for performing tasks during the test.

### Embodiment 28

The method of any one of embodiments 16 to 27, wherein said data indicative for spatial working memory comprise data from a computer-implemented test for working memory.

### Embodiment 29

The method of any one of embodiments 16 to 28, wherein said data indicative for cooperation behavior comprise data from a computer-implemented test assessing cooperation behavior.

### Embodiment 30

The method of any one of embodiments 16 to 29, wherein said data indicative for image exploration capabilities, vocal properties and speaker recognition comprise data from a computer-implemented test for visually identifying social and non-social elements, voice characteristics, and/or speaker recognition by conversation and ambient sound.

### Embodiment 31

The method of any one of embodiments 16 to 30, wherein said dataset comprising behavior data comprises at least:
(i) data indicative for conversational skills and obsessive interest;
(iv) data indicative for sleep behavior;
(v) data indicative for anxiety;
(vi) data indicative for emotion recognition;
(vii) data indicative for spatial working memory;
(viii) data indicative for cooperation behavior; and
(ix) data indicative for image exploration capabilities, vocal properties and speaker recognition
   or
(i) data indicative for conversational skills and obsessive interest;
(ii) data indicative for sociability and routines;
(iii) data indicative for repetitive movements;
(v) data indicative for anxiety;
(vi) data indicative for emotion recognition;
(vii) data indicative for spatial working memory;
(viii) data indicative for cooperation behavior; and
(ix) data indicative for image exploration capabilities, vocal properties and speaker recognition.

### Embodiment 32

The method of any one of embodiments 16 to 31, wherein said subject is a human.

### Embodiment 33

A method for recommending a therapy for ASD comprising the steps of:
(a) assessing ASD by carrying out the method of any one of embodiments 16 to 32; and
(b) recommending a therapy for ASD based on the assessment provided in step (a).

### Embodiment 34

A method for treating and/or preventing ASD in a subject suffering or suspect to suffer therefrom comprising
(a) assessing ASD by carrying out the method of any one of embodiments 16 to 32; and
(b) applying a therapy for ASD based on the assessment provided in step (a).

### Embodiment 35

The method of embodiment 33 or 34, wherein said therapy for ASD comprises treatment by at least one drug selected from the group consisting of: a Vasopressin 1a antagonist, more particularly Balovaptan, a N-Methyl-D-Aspartate (NMDA) receptor antagonists, in particular memantine or RVT-701, a selective inhibitor of the enzyme fatty acid amide hydrolase (FAAH), in particular JNJ-5279, a GABA-modulator, in particular a GABA Aa5 positive allosteric modulator (PAM), in particular RG7816, a GABA A modulator or a selective GABA-B agonist, in particular arbaclofen, a mGlu4/7 positive allosteric modulator, oxytocin, in particular OPN-300, a Acetyl-Choline Esterase Inhibitor, in particular donepezil, a dual inhibitor of lysine (K)-specific demethylase 1A/monoamine oxidase B, in particular Vafidemstat, a tyrosine hydroxylase inhibitor, in particular L1-79, a selective and irreversible small molecule non-ATP-competitive glycogen synthase kinase 3 (GSK-3) inhibitor, in particular Tideglusib, an Amylase, lipase & protease regulator enzymes like CM-AT, a NKCC1 cation-chloride co-transporter blocker, in particular bumetamide, a microbiota transfer therapy, in particular FSM®, a microbiome modulator, in particular AB-2004, a selective serotonin reuptake inhibitor, in particular fluoxetine, a dopamine 2 receptor antagonist, in particular risperidone, ziprasidone or lurasidone, a non-euphoric cannabinoid, in particular Zygel, a phytocannabinoid, in particular Cannabidivarin, a mu-opioid receptor antagonist, in particular naloxone or naltrexone or Fatty Acids Omega-3 or folinic acid treatment, in a particular sub embodiment of embodiment 35, a Vasopressin 1a antagonist, more particularly Balovaptan, a GABA-Aa5 PAM, a GABA-A modulator, a mGlu4/7 PAM, a Dopamine 2 receptor antagonist, in particular Risperidone, mu-opioid receptor antagonist, in particular naloxone, and NMDA glutamate receptor antagonist, in particular memantine.

### Embodiment 36

A mobile device comprising a processor, at least one sensor recording behavior data and a database as well as software which is tangibly embedded to said device and, when running on said device, carries out the method of any one of embodiments 16 to 34.

### Embodiment 37

A system comprising a mobile device comprising at least one sensor recording behavior data and a remote device comprising a processor and a database as well as software which is tangibly embedded to said device and, when running on said device, carries out the method of any one of embodiments 16 to 34, wherein said mobile device and said remote device are operatively linked to each other.

### Embodiment 38

Use of the mobile device according to embodiment 36 or the system of embodiment 37 for assessing ASD.

### Embodiment 39

A method assessing an autism spectrum disorder (ASD) in a subject comprising the steps of:
a) determining at least one usage behavior parameter from a dataset comprising usage data for a mobile device within a first predefined time window wherein said mobile device has been used by the subject; and
b) comparing the determined at least one usage behavior parameter to a reference, whereby an autism spectrum disorder will be assessed.

### Embodiment 40

The method of embodiment 39, wherein said assessing an autism spectrum disorder comprises assessing at least one negative symptom associated with an autism spectrum disorder selected from the group consisting of: social communication and social interaction, and restricted, repetitive patterns of behavior, interests or activities.

### Embodiment 41

The method of embodiment 40, wherein said assessing an autism spectrum disorder comprises determining an improvement of the at least one negative symptom associated with an autism spectrum disorder.

### Embodiment 42

The method of any one of embodiments 39 to 41, wherein the said usage data for a mobile device comprise data selected from the group consisting of: phone usage data, application (App) usage data, ambient noise data, movement capture data and location capture data.

### Embodiment 43

The method of any one of embodiments 39 to 42, wherein said at least one usage behavior parameter is a recorded variable selected from the list consisting of
(i) phone and/or app usage, in an embodiment logged contacts, logged calls, logged SMS, logged app usage, logged screen on, and/or logged WIFI & bluetooth;
(ii) ambient sound, in an embodiment volume, time and/or pitch of ambient sound, and/or frequency, time, and/or duration of speech;
(iii) movement, in an embodiment activity levels and/or location data;
(iv) light and proximity data, in an embodiment phone handling;
(v) touch behavior, in an embodiment touch interactions and/or typing behavior.

### Embodiment 44

The method of embodiment 43, wherein an improvement of at least one negative symptom associated with an autism spectrum disorder is determined if the at least one usage behavior parameter improves as follows in the case of an autism spectrum disorder:
(i) phone and/or app usage: increased number of contacts called, increased phone call duration and increased number of characters in SMS, decreased time and frequency of non-social apps and/or games, increase in frequency and time spent in social apps, decrease of the total amount of time spent using Apps, decreased unlock duration every time the patient uses the phone, increased number of networks (WIFI) and devices (bluetooth) during the day, decrease of duration connected to the most used network (home), and/or increased duration connected to networks different from the most used network;
(ii) ambient sound: increased volume during the day, larger increases of ambient sound during the morning, higher pitch in voiced frames, increased ratio of voiced to non-voiced frames, increased duration in the voiced frames, and/or more time spent in social places;
(iii) movement: increased activity during the day, decreased activity during the night, increased walk duration, longer walks, decreased duration of not moving, increased time on car travels, increased number of new places visited, longer distances covered during the day, and/or reduced time spent in a single place (home);
(iv) light and proximity data: increased duration of the phone in the pocket, and/or decreased duration of use of the phone in the darkness; and/or
(v) touch behavior: decreased activity and interaction in non-social apps and/or games, increased interaction with social apps; less browsing behavior in Apps, as measured by swipe gestures; changes to the circadian rhythm, in an embodiment less interactions at night/in darkness; increased amounts of typing behavior; increased amounts of typing behavior in social apps; increased use of certain punctuation marks, in an embodiment question marks and exclamation marks; faster typing behavior.

### Embodiment 45

The method of any one of embodiments 39 to 44, wherein said reference is at least one usage behavior parameter which has been determined in a dataset comprising usage data for a mobile device within a second predefined time window prior to the first predefined time window.

### Embodiment 46

The method of embodiment 45, wherein between the second and the first time window the subject has received an autism spectrum disorder therapy or a therapy for at least of the negative symptoms associated therewith.

### Embodiment 47

The method of embodiment 46, wherein said therapy is a drug-based therapy.

### Embodiment 48

The method of embodiment 46 or 47, wherein an improvement of at least one negative symptom associated with schizophrenia or an autism spectrum disorder is indicative for a successful therapy.

### Embodiment 49

The method of any one of embodiments 39 to 48, wherein said behavior data comprise one or more data selected from the group consisting of:
(i) data indicative for conversational skills and obsessive interest;
(ii) data indicative for sociability and routines;
(iii) data indicative for repetitive movements;
(iv) data indicative for sleep behavior;
(v) data indicative for anxiety;
(vi) data indicative for emotion recognition;
(vii) data indicative for spatial working memory;
(viii) data indicative for cooperation behavior; and
(ix) data indicative for image exploration capabilities, vocal properties and speaker recognition.

### Embodiment 50

The method of any one of embodiments 39 to 49, wherein said assessing ASD comprises assessing at least one symptom associated with an autism spectrum disorder selected from the group consisting of: social communication and social interaction, and restricted, repetitive patterns of behavior, interests or activities.

### Embodiment 51

The method embodiment 50, wherein said data indicative for conversational skills and obsessive interest comprise data for voice characteristics, amount of speech and/or turn-taking behavior during conversations.

### Embodiment 52

The method of embodiment 50 or 51, wherein said data indicative for repetitive movements comprise data for frequency and duration of repetitive and/or stereotype movements.

### Embodiment 53

The method of any one of embodiments 50 to 52, wherein said data indicative for sleep behavior comprise data for sleep latency, sleep efficiency, sleep time, waking after sleep onset and/or sleepiness.

### Embodiment 54

The method of any one of embodiments 50 to 53, wherein said data indicative for anxiety comprise data for heart rate variability.

### Embodiment 55

The method of any one of embodiments 50 to 54, wherein said data indicative for emotion recognition comprise data from a computer-implemented reading the mind in the eyes test (RMET), in particular, emotional intensity for recognizing emotions simulated by tasks in the test, response and decision time for performing tasks during the test.

### Embodiment 56

The method of any one of embodiments 50 to 55, wherein said data indicative for spatial working memory comprise data from a computer-implemented test for working memory.

### Embodiment 57

The method of any one of embodiments 50 to 56, wherein said data indicative for cooperation behavior comprise data from a computer-implemented test assessing cooperation behavior.

### Embodiment 58

The method of any one of embodiments 50 to 57, wherein said data indicative for image exploration capabilities, vocal properties and speaker recognition comprise data from a computer-implemented test for visually identifying social and non-social elements, voice characteristics, and/or speaker recognition by conversation and ambient sound.

### Embodiment 59

The method of any one of embodiments 39 to 58, wherein said mobile device is a smartphone, smartwatch, wearable sensor, portable multimedia device or tablet computer.

### Embodiment 60

The method of any one of embodiments 39 to 59, wherein said subject is a human.

### Embodiment 61

A mobile device comprising a processor, at least one sensor recording usage data and a database as well as software which is tangibly embedded to said device and, when running on said device, carries out the method of any one of embodiments 39 to 60.

### Embodiment 62

A system comprising a mobile device comprising at least one sensor recording usage data and a remote device comprising a processor and a database as well as software which is tangibly embedded to said device and, when running on said device, carries out the method of any one of embodiments 39 to 60, wherein said mobile device and said remote device are operatively linked to each other.

### Embodiment 63

Use of the mobile device according to embodiment 61 or the system of embodiment 62 for assessing schizophrenia or an autism spectrum disorder by analyzing a dataset comprising usage data for a mobile device within a first predefined time window wherein said mobile device has been used by the subject.

### Embodiment 64

A method for recommending a therapy for ASD comprising the steps of:
(a) assessing ASD by carrying out the method of any one of embodiments 39 to 60; and
(b) recommending a therapy for ASD based on the assessment provided in step (a), wherein said therapy for ASD is, typically, treatment by at least one drug selected from the group consisting of: a Vasopressin 1a antagonist, more particularly Balovaptan, a N-Methyl-D-Aspartate (NMDA) receptor antagonists, in particular memantine or RVT-701, a selective inhibitor of the enzyme fatty acid amide hydrolase (FAAH), in particular JNJ-5279, a GABA-modulator, in particular a GABA Aa5 positive allosteric modulator (PAM), in particular RG7816, a GABA A modulator or a selective GABA-B agonist, in particular arbaclofen, a mGlu4/7 positive allosteric modulator, oxytocin, in particular OPN-300, a Acetyl-Choline Esterase Inhibitor, in particular donepezil, a dual inhibitor of lysine (K)-specific demethylase 1A/monoamine oxidase B, in particular Vafidemstat, a tyrosine hydroxylase inhibitor, in particular L1-79, a selective and irreversible small molecule non-ATP-competitive glycogen synthase kinase 3 (GSK-3) inhibitor, in particular Tideglusib, an Amylase, lipase & protease regulator enzymes like CM-AT, a NKCC1 cation-chloride co-transporter blocker, in particular bumetamide, a microbiota transfer therapy, in particular FSM®, a microbiome modulator, in particular AB-2004, a selective serotonin reuptake inhibitor, in particular fluoxetine, a dopamine 2 receptor antagonist, in particular risperidone, ziprasidone or lurasidone, a non-euphoric cannabinoid, in particular Zygel, a phytocannabinoid, in particular Cannabidivarin, a mu-opioid receptor antagonist, in particular naloxone or naltrexone or Fatty Acids Omega-3 or folinic acid treatment, in a particular a drug selected from, a Vasopressin 1a antagonist, more particularly Balovaptan, a GABA-Aa5 PAM, a GABA-A modulator, a mGlu4/7 PAM, a Dopamine 2 receptor antagonist, in particular Risperidone, mu-opioid receptor antagonist, in particular naloxone, and NMDA glutamate receptor antagonist, in particular memantine.

All references cited throughout this specification are herewith incorporated by reference with respect to the specific disclosure content referred to as well as in their entireties.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1A shows the activation of the App for data capture from patients informing the patient about the usage behavior that will be captured;

FIG. 1B shows the capture of contacts;

FIG. 1C shows phone calls and messages;

FIG. 1D shows App usage;

FIG. 1E shows ambient noise;

FIG. 1F shows location and movement;

FIG. 1G shows that the App will inform how data capture can be stopped or interrupted.

FIG. 2A shows a profile of captured phone usage data from a patient.

FIG. 2B shows a profile of captured app usage data from a patient.

FIG. 2C shows a profile of captured accelerometer data from a patient.

FIG. 2D shows a profile of captured ambient noise data from a patient.

FIG. 3 shows behavioral parameters for emotion recognition, in particular, behavioral parameters from facial expressions.

FIG. 4 shows behavioral parameters for sociability and routines, in particular, behavioral parameters from beacons.

The data shown in FIGS. 5A and 5B concerns the conversations recorded using the Conversation Task in the ASD App. The number of conversation recordings per week in the study conducted by the participants and the Duration of the Conversation Recordings is shown.

FIG. 5A shows the probability density plots for the number of conversations recorded per week in study by participants.

FIG. 5B shows the probability distribution for duration of collected audio recordings.

FIGS. 6A and 6B show the conversation statistics recorded in the ASD App. The average duration of study participant responses (response duration; FIG. 6A) and the amount of time spent speaking by the participant (proportion participant; FIG. 6B) differentiate between known study groups (*: p<0.05, **: p>0.005, ***: p<0.0005). Shown are data of Low-Functioning ASD patients (LF), High Functioning ASD patients (HF) and Control groups (TD: “typical developing”). The response duration and the amount of time speaking is depicted.

FIG. 7 shows the facial expression task recorded in the ASD App. The intensity of emotion (overall morph level) is depicted. The overall morph feature (Intensity of emotion presented) discriminates between cohort groups. It is demonstrated that Control (TD) and ASD HF Adults and ASD HF Children require less emotional intensity presented for discrimination of the displayed emotion.

### DESCRIPTION AND EXAMPLES

The embodiments and examples described below are not intended to be exhaustive or to limit the invention to the precise forms disclosed in the following detailed description. Rather, the embodiments are chosen and described so that others skilled in the art may appreciate and understand the principles and practices of this disclosure.

### Example 1: Investigation of Mobile Phone Behavior Over 16 Weeks in Schizophrenia Patients

The smart phone usage behavior of 100 patients suffering from schizophrenia will be monitored over a period of 16 weeks (observation period). The patients will use Android based smart phones. Patients may receive a drug. Smart phone usage which will be investigated includes phone usages, App usage, ambient noise, movement, location and general handling as well as touch behavior.

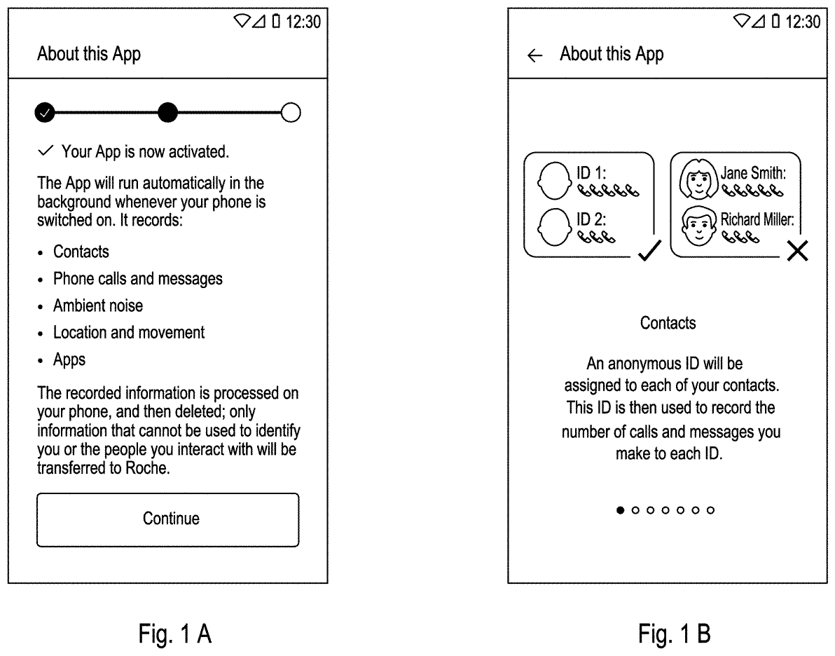

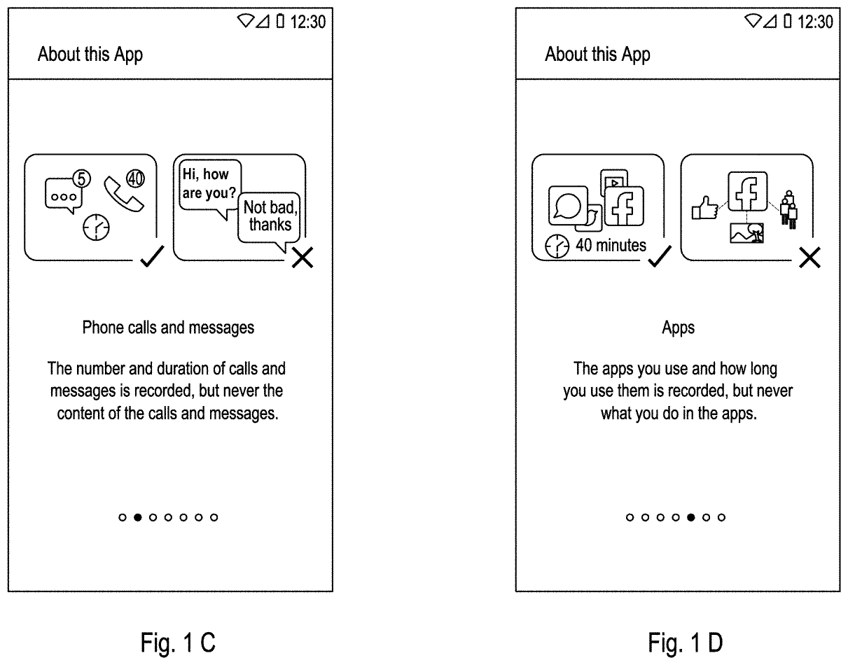

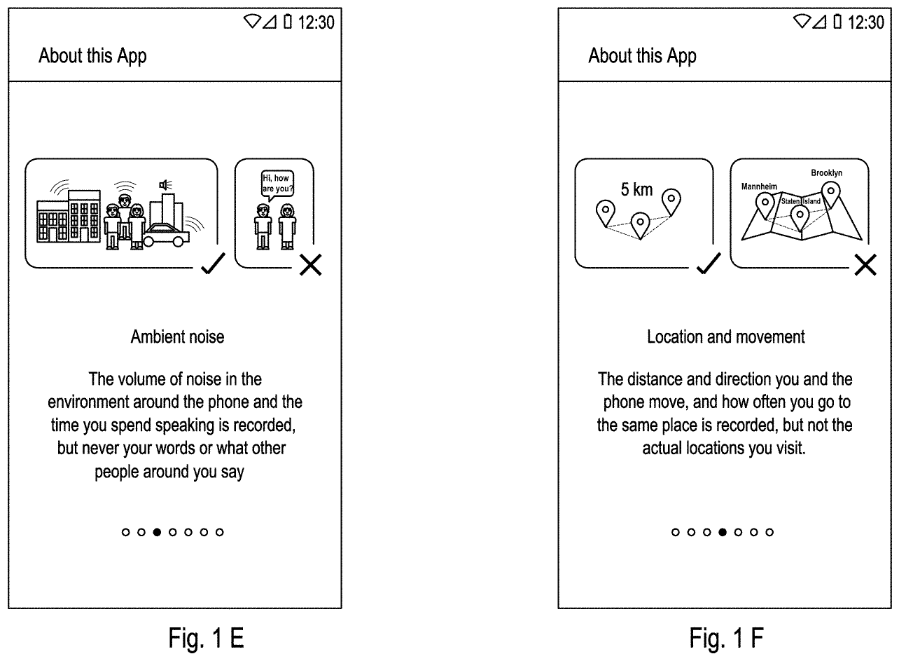

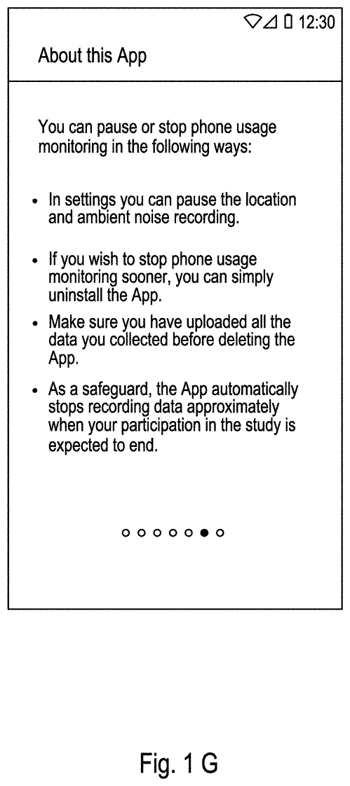

In order to capture the said usage data, an App will be installed on the smart phones of the patients. The App will automatically capture the usage behavior data within a certain time window, derive usage behavior parameters therefrom and store these parameters on the smart phone. The data capture will be carried out several times during the observation period, e.g., each day. The App will inform the patient once data capture is started and when it ends (FIG. 1). Moreover, in order to safeguard data protection provisions, the App will be activated by an investigator at the beginning of the observation period and de-installed by the said investigator at the end of the observation period. Only patients which have given their informed consent will be observed. All data which may be transferred during before, during or after the observation period will be encrypted.

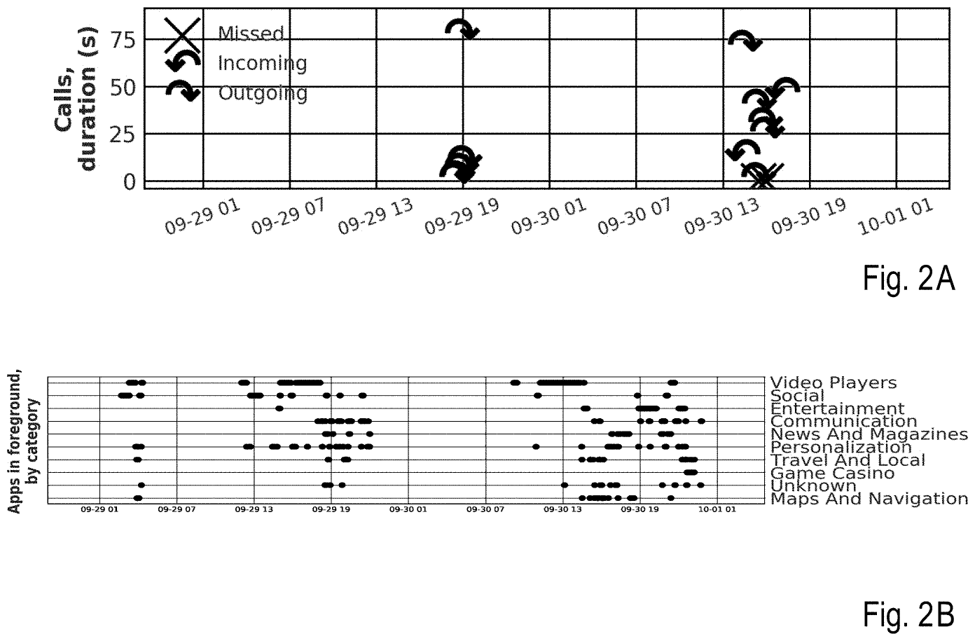

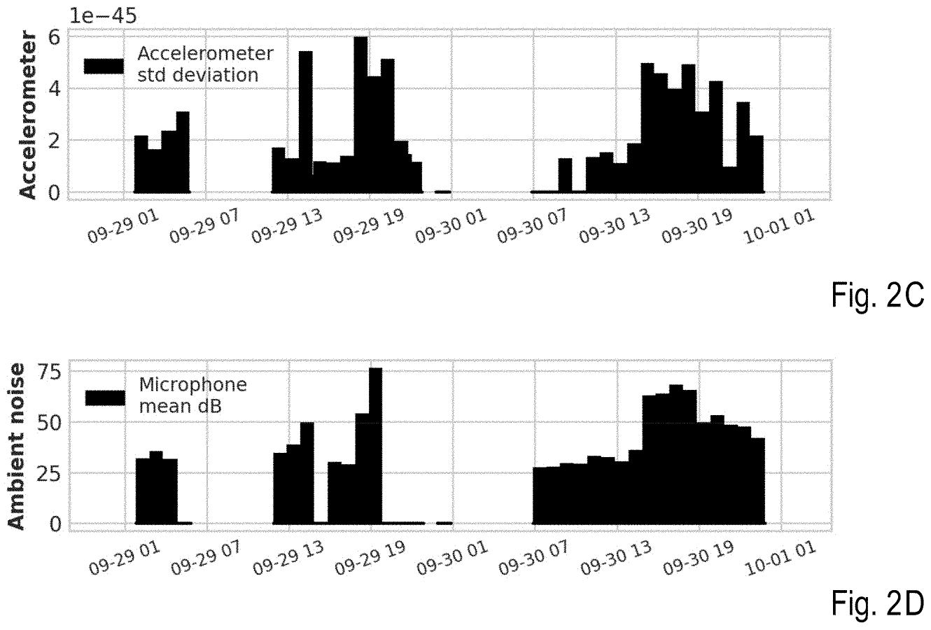

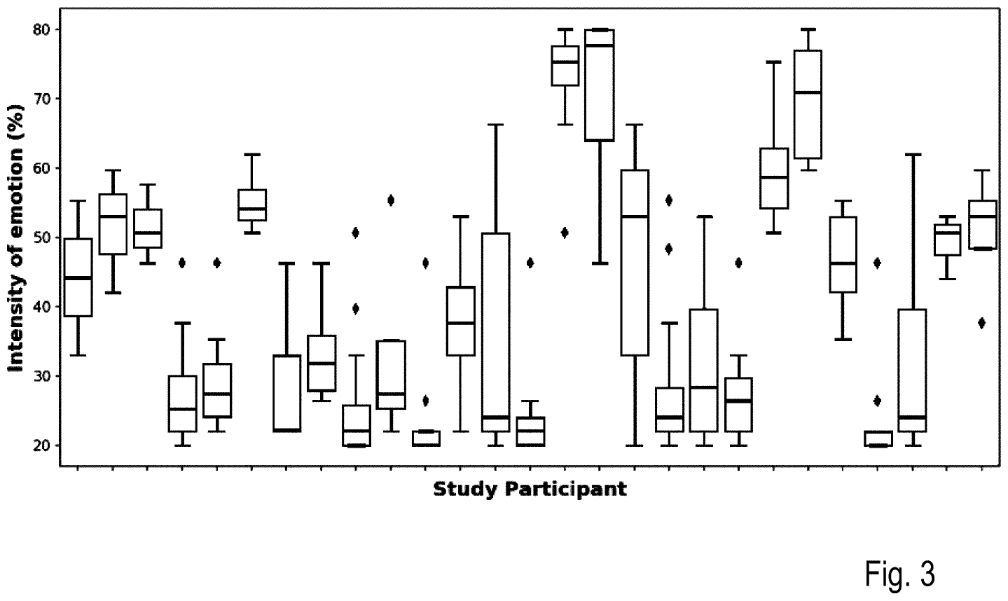

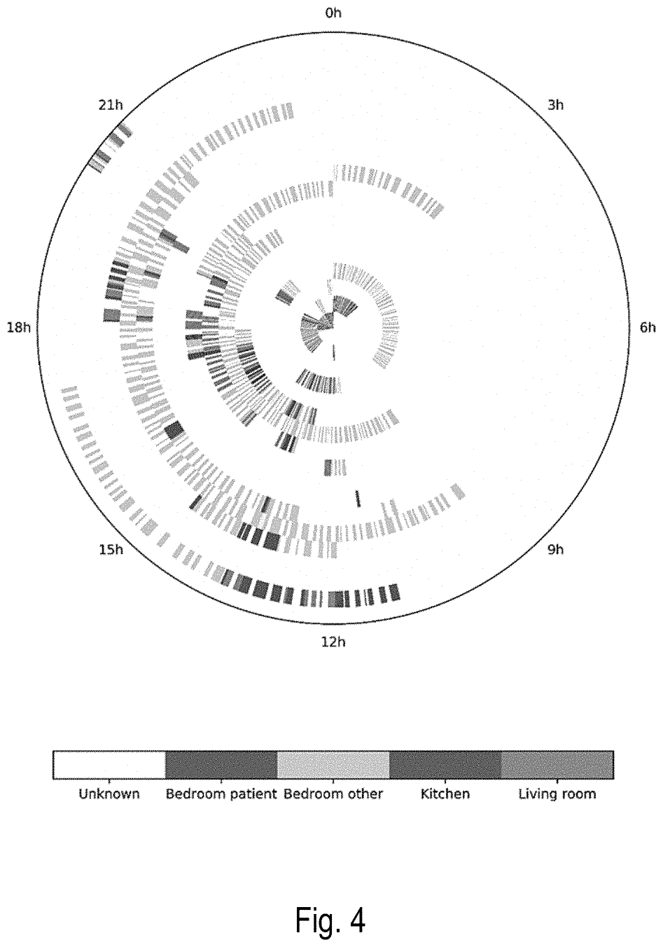

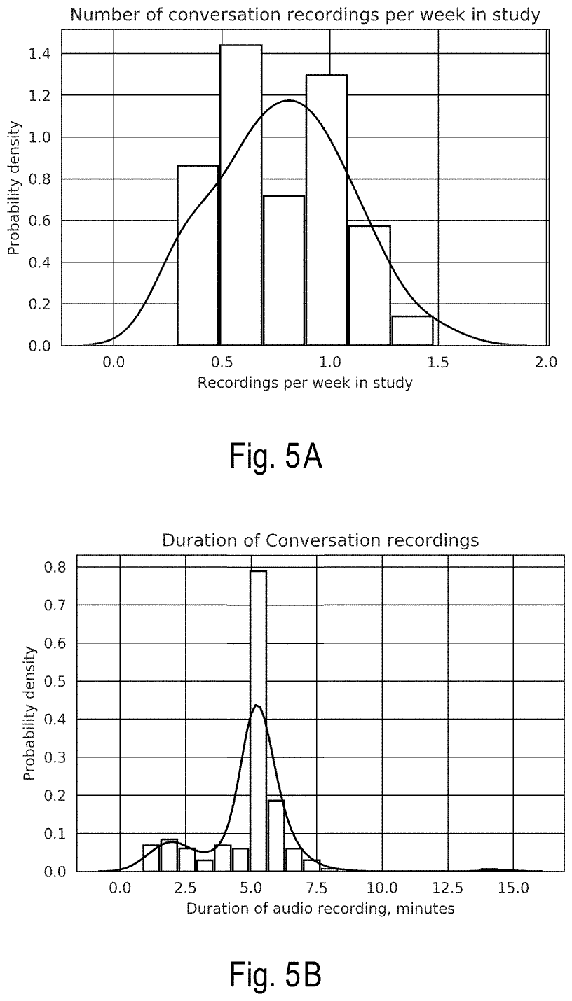

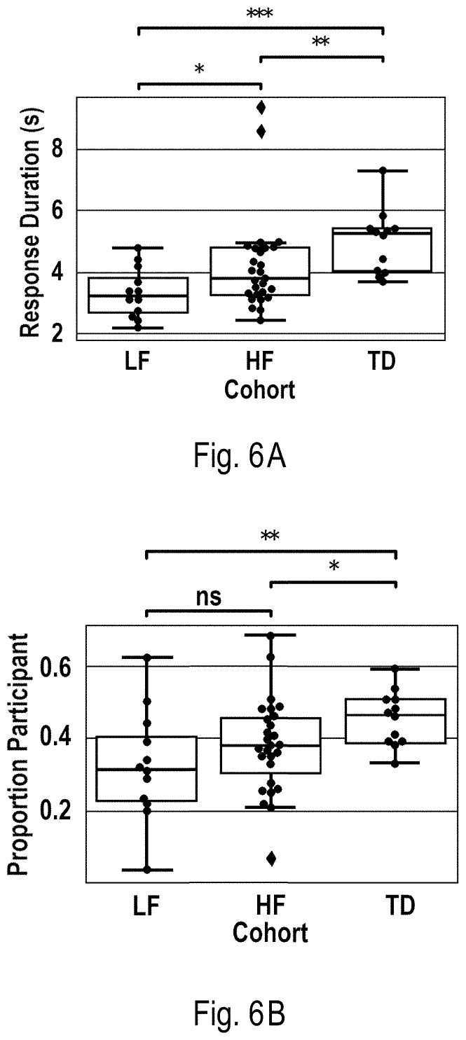

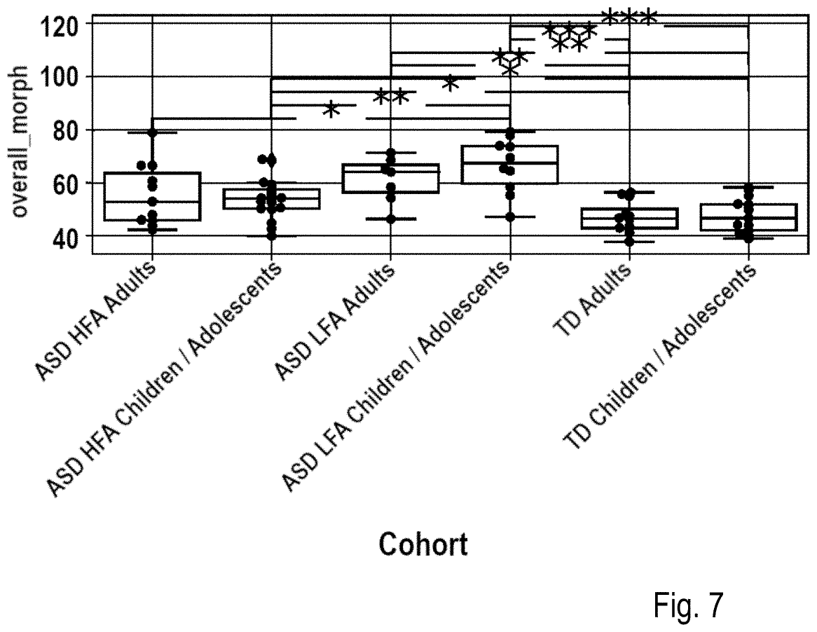

A profile of captured data from a patient is depicted in FIG. 2.

### Example 2: Investigation of Mobile Phone Behavior Over 16 Weeks in ASD Patients

The smart phone usage behavior of 100 patients suffering from an autism spectrum disorder will be monitored over a period of 16 weeks (observation period). The patients will use Android based smart phones. Patients may receive a drug. Smart phone usage which will be investigated includes phone usages, App usage, ambient noise, movement, location and general handling as well as touch behavior.

In order to capture the said usage data, an App will be installed on the smart phones of the patients. The App will automatically capture the usage behavior data within a certain time window, derive usage behavior parameters therefrom as indicated in Tables 1 and 2 and store these parameters on the smart phone. The data capture will be carried out several times during the observation period, e.g., each day. The App will inform the patient once data capture is started and when it ends (FIG. 1). Moreover, in order to safeguard data protection provisions, the App will be activated by an investigator at the beginning of the observation period and de-installed by the said investigator at the end of the observation period. Only patients which have given their informed consent will be observed. All data which may be transferred during before, during or after the observation period will be encrypted.

### Example 3: Behavior Data Acquisition by Smart Watches and/or Smart Phones

Smart watches and/or smart phones were equipped to measure the following behavior data from patients.

For Conversational Skills and Obsessive Interest, the Following Test was Implemented:

Background: Individuals with ASD can have unusual vocal properties, a reduced amount of speech and difficulty with turn-taking. They may intensely focus on their restricted interest making conversations difficult. 66% of individuals with ASD have a history of aggressive episodes.

Method: Support person records weekly conversation with participant Features that allow subsequent spectral, semantic and sentiment analyses are extracted and uploaded.

Example Metrics: Characteristics of voice (pitch, volume, shimmer, jitter); Turn-taking behavior during conversation; Repeated reference to same topic.

For Sociability and Routines, the Following Test was Implemented:

Background: Individuals with ASD are less likely to engage in social approaches and to interact with others than non-ASD individuals.

Method: Rooms of home are labelled as social/non-social/sometimes social. Bluetooth transmitters placed in these rooms and carried by household. Distance between smartwatch (worn by participant) and transmitters is estimated to identify time in social rooms and around others.

Example Metrics: Time in social vs. non-social rooms; Time close to other people in the home.

For Repetitive Movements, the Following Test was Implemented:

Background: Individuals with ASD often have repetitive movements such as hand-flapping and body rocking. Recent studies have demonstrated the potential for automated detection of repetitive movements.

Method: Study participant's movement is tracked with smartwatch. When support person sees study participant performing a repetitive movement, they log a timestamp using a wearable movement logger or a function in the smartphone app. Algorithm learns patterns of sensor data associated with repetitive movements and tracks these events during everyday life.

Example Metrics: Frequency and duration of repetitive movement types.

For Sleep Behavior, the Following Test was Implemented:

Background: Individuals with ASD can have difficulty sleeping, reflected in longer sleep latencies and more difficulty going to bed and falling asleep.

Method: Two nights per week participant wears smartwatch overnight. Sleep patterns are extracted based on body movement data from watch. Participants complete an electronic patient reported outcome sleep diary every four days.

Example Metrics: Time to sleep onset; Sleep duration.

For Anxiety, the Following Test was Implemented:

Background: The co-morbidity of anxiety disorders with ASD is estimated to be 39.6%. Anxiety is associated with lower rates of heart-rate variability.

Method: Smartwatch captures PPG signal throughout the day. Location is captured based on indoor location tracking, using Beacon technology. Social situations and routine changes inferred from location data. PPG signal is used to estimate heart rate variability. Anxiety ratings are captured with an ecological momentary assessment.

Example Metrics: Heart-rate variability when in social locations and on days with unusual routine; Association between anxiety ratings and heart-rate variability.

For Emotion Recognition, the Following Test was Implemented:

Background: The Reading the Mind in the Eyes Test (RMET) is a well established assessment of the ability to recognize the mental states of others and was adapted for a smartphone use.

Method: Participant shown static image of a facial expression. Intensity of emotion on face varies on a trial-by-trial basis according to an adaptive algorithm. Participant must tap on screen when they recognize emotion. Participant labels emotion.

Example Metrics: Emotional intensity at which participant recognizes emotion; Response time; Decision time.

For Spatial Working Memory, the Following Test was Implemented:

Background: Individuals with autism can have difficulty with working memory. They are more likely to make errors than non-ASD individuals on the CANTAB assessment of spatial working memory, and are less likely to consistently use a specific organized search strategy.

Method: In this task, the participant must remember which chickens have laid eggs. Participant can search for eggs by tapping on a chicken to check if it has laid one. Once a chicken has laid an egg they will not lay another, so the participant should not re-check that chicken. They should also not check the same chicken twice within one search. Difficulty levels: 4, 6, 8, 10, 12 chickens.

Example Metrics: Number of times chicken is checked twice in same search; Number of times chicken is checked that already laid egg.

For Cooperation Behavior, the Following Test was Implemented:

Background: “Stag Hunt” (named Treasure Hunt for this app) was developed to assess the cooperative ability of individuals with ASD. Difficulty in representing the strategy of another player has been shown to predict symptom severity.

Method: Participant plays turn-taking game with a computer agent. Participant can either:

Pursue a coin, worth $1, which can be captured alone. Pursue treasure chest, worth $4; which requires working in cooperation with computer agent.

Example Metrics: Percent of times participant chooses to cooperate; Points gained when cooperating.

For Image Exploration Capabilities, Vocal Properties and Speaker Recognition, the Following Test was Implemented:

Background: People with ASD show distinctive, atypical acoustic patterns of speech and a tendency to fixate on non-social elements of images, such as those used in the ADOS.

Method: Participant is asked to communicate what is happening in a picture that contains social and non social elements. Voice is recorded, as is image browsing behavior.

Example Metrics: Finger motion tracking provides proxy for gazing behavior, indicating time spent inspecting social or non-social elements of image; Characteristics of voice (pitch, volume, shimmer, jitter); In addition, acoustic fingerprint is extracted for speaker identification in Conversation data.

### Example 4: Investigation of Behavior of 59 Participants Using Smart Watches

The behavior of 59 participants was monitored. Almost all participants were willing to do the tasks in the context of a clinical trial. The Smartwatch was well-received in terms of design and comfort. Almost all participants were open to using Beacons in their home, without privacy or feasibility concerns. Only minor usability issues were observed, which were addressed with modifications to active tasks. Feasibility of performing tasks depended on age and IQ—addressed by adding option for healthcare professional to deactivate task.

The data capture was carried out several times during the observation period.

Results for emotion recognition is shown in FIG. 1. Participants were asked to identify the emotion presented in a series of photos of facial expressions with different emotional intensities. If the participant responds correctly or incorrectly, then the next time that the emotion is displayed the intensity reduces or increases, respectively. It is expected that an emotion detection threshold will be reached at which the participant correctly identifies the emotion on ˜50% of trials. This figure presents data from 28 individuals with ASD. On each box the central mark indicates the median happiness intensity, and the bottom and top edges of the box indicate the 25th and 75th percentiles, respectively. The whiskers extend to the most extreme data points not considered outliers, and the outliers are plotted individually using the diamond symbol. The median intensity varied across participants suggesting the task is sensitive to different emotion detection thresholds.

Results for sociability and routines are shown in FIG. 2. Participants were asked to place Beacons around their home. The Beacons emitted a Bluetooth signal with an ID that is associated with a room. The Bluetooth signal strength, captured by the participant's smartwatch, was used to estimate which room the participant is in. This figure indicates this method can successfully identify the room location of the participant based on the Beacon data. Each ring represents a day in the life of an individual with ASD (age between 5 and 12 years, IQ>=70), with colored markings indicating the room where it is estimated the participant was located at that time. Grey areas indicate no Beacon data is available (watch is switched off or participant is not in range of any Beacons).

TABLE 1
Data for phone usage and ambient sound

| | | | Why we are recording this: |
|---|---|---|---|
| | | | We expect that patients with |
| | | | improvements in Autism Spectrum |
| | | | Disorder Sociability and |
| | | | Communication domains of clinical |
| | | | scales that measure sociability (SRS-2, |
| Domain | Sub-domain | Variables being recorded | ADOS-2, VINELAND-II) will show... |
| Phone | Anonymous ID generated for contacts, name, number and photo ID. This | | |
| and App | table is stored in device storage only. | | |
| Usage | Log | Each contact is assigned an | Increased the number of contacts they |
| | Contacts | anonymous ID. Calls and | call, phone call duration and number |
| | | SMS are logged against | of characters |
| | | this ID (see below) | |
| | Log Calls | Frequency, time, duration, | |
| | | incoming or outgoing | |
| | Log SMS | Frequency, time, duration, | |
| | | incoming or outgoing, | |
| | | number of characters | |
| | Log App | Name of App | Decreased the time and frequency of |
| | Usage | Frequency, time, duration | non-social apps and/or games, while |
| | | of App usage | increasing the frequency and time |
| | | (foreground/background) | spend in Social apps. |
| | | | Overall, we expect the total amount of |
| | | | time spend using App will decrease. |
| | Log Screen | Frequency, time, duration | Decreased unlock duration every time |
| | On | | the patient use the phone |
| | Log WIFI | Number of visible WIFI & | Increased number of networks (WIFI) |
| | & bluetooth | Bluetooth | and devices (bluetooth) during the day |
| | | Number of WIFIs used | Decrease duration connected to the |
| | | | most used network (home) |
| | | | Increased duration connected to |
| | | | different networks |
| Ambient | Audio is recorded for 10 seconds every minute, processed on the phone | | |
| Sound | to compute the features below. Occurs in memory and is never stored. | | |
| | The raw audio recordings are discarded once the features are computed. | | |
| | Volume & | Volume (power), time | Increased volume during the day, but |
| | pitch | | larger increases during the morning |
| | | | Higher pitch in voiced frames |
| | Speech | Frequency, time, duration | Increased ratio of voiced and non- |
| | Classifier | | voiced frames |
| | | | Increased duration in the voiced frames |
| | Sound | Mel frequency Cepstral | (Required for further optimizing the |
| | power | Coefficients | speech classifier) |
| | spectrum | | |

TABLE 2
Data for movement and light & proximity

| | | | Why we are recording this: |
|---|---|---|---|
| | | | We expect that patients with |
| | | | improvements in Autism Spectrum |
| | | | Disorder Sociability and |
| | | | Communication domains of clinical |
| | | | scales that measure sociability (SRS-2, |
| Domain | Sub-domain | Variables being recorded | ADOS, VINELAND-II) will show... |
| Movement | Activity | Tri-axial acceleration | Increased activity during the day |
| | Levels | (20 Hz), time | Decreased activity during the night |
| | | | Using motor behavior classification: |
| | | | Increased walk duration, longer walks |
| | | | Decreased duration of not moving |
| | | | Increased time on car travels |
| | Location | Obfuscated GPS, i.e., | Increased number of new places |
| | | distance and direction | visited |
| | | of travel | More time spent in social places, |
| | | | identified using ambient noise |
| | | | measures (please also add for |
| | | | Schizophrenia) |
| | | | Longer distance covered during the |
| | | | day |
| | | | Reduced time spend in a single place |
| | | | (home) |
| Light & | Phone | Amount of ambient | Increased duration of the phone in the |
| proximity | handling | light over time | pocket |
| | classification | Proximity of objects | Decreased duration of use of the |
| | | over time | phone in the darkness |
| Phone | Technical | Android version of the | For technical diagnostics only |
| information | phone | device | |
| | information | Battery Health | |
| | | Battery consumption | |
| | | Storage Space Total and | |
| | | consumed (intenal and | |
| | | SD card) | |
| | | Data size (study and | |
| | | non-study related) | |

TABLE 3
Data from touch behavior

| | | | Why we are recording this: |
|---|---|---|---|
| | | | We expect that patients with |
| | | | improvements in Autism Spectrum |
| | | | Disorder Sociability and |
| | | | Communication domains of clinical |
| | | | scales that measure sociability |
| | | | (SRS-2, ADOS, VINELAND-II) |
| Domain | Sub-domain | Variables being recorded | will show... |
| Touch | Touch | For every touch interaction: | Decreased amount of activity and |
| behavior | interactions | Touch down, swiping and | interaction in non-social apps |
| | | touch up | and/or games, while increased |
| | | Length and directionality of | interaction with social apps. |
| | | the touch movement | Less browsing behavior in Apps, |
| | | Y-coordinate of the touch | as measured by swipe gestures |
| | | event only | Changes to the circadian rhythm, |
| | | Time stamps | i.e., less interactions at night/in |
| | | Whether it occurred on the | darkness |
| | | keyboard | |
| | Typing | For all characters entered on | Increased amounts of typing |
| | behavior | the screen via the keyboard: | behavior |
| | | Character type (letter, number, | Increased amounts of typing |
| | | punctuation mark, editing | behavior in social apps |
| | | characters, function key, emoji) | Increased used of certain |
| | | Actual character used only for | punctuation marks, e.g., question |
| | | the following character types: | marks and exclamation marks |
| | | punctuation mark (e.g., full | Faster typing behavior |
| | | stops, exclamation marks, | Changes to the circadian rhythm, |
| | | editing characters (e.g., space, | i.e., less interactions at night/in |
| | | delete, backspace) | darkness |
| | | Time stamps | |

TABLE 4
Behavior parameters in ASD

| Behavior (domain) | parameters | expectations |
|---|---|---|
| conversational | voice characteristics (pitch, | irregularties |
| skills and obsessive | volume, shimmer and jitter), | |
| interest | amount of speech, | reduced |
| | turn-taking behavior during | difficulties |
| | conversations | |
| sociability and | time in social versus non-social | reduced |
| routines | rooms, | |
| | time in proximity to other people | reduced |
| repetitive | frequency of repetitive and/or | increased |
| movements | stereotype movements, | |
| | duration of repetitive and/or | increased |
| | stereotype movements | |
| sleep behavior | sleep latency, | Larger |
| | sleep efficiency, | reduced |
| | sleep time, | reduced |
| | waking after sleep onset, | increased |
| | sleepiness | increased |
| anxiety | heart rate variability | lowered |
| emotion | emotional intensity for recognizing | reduced |
| recognition | emotions simulated by tasks in the | |
| | RMET test, | |
| | response and decision time for | reduced |
| | performing tasks in the RMET test | |
| spatial working | trials for performing a memory- | increased |
| memory | dependent task | |
| cooperation | number of cooperation events | reduced |
| behavior | when performing a task | |
| image exploration | social and non-social elements, | focus on non- |
| capabilities, vocal | | social elements |
| properties and | | for longer time |
| speaker recognition | voice characteristics | irregularities |

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

What is claimed is:

1. A method of assessing an autism spectrum disorder (ASD) in a subject, comprising:
a) determining at least one usage behavior parameter from a dataset comprising usage data for a mobile device within a first predefined time window wherein said mobile device has been used by the subject;
b) comparing the determined at least one usage behavior parameter to a reference; and
c) assessing autism spectrum disorder in the subject based on the comparison of step b).

2. The method of claim 1, wherein said assessing an autism spectrum disorder comprises assessing at least one negative symptom associated with an autism spectrum disorder selected from the group consisting of: social communication and social interaction; and restricted, repetitive patterns of behavior, interests or activities.

3. The method of claim 2, wherein said assessing an autism spectrum disorder comprises determining an improvement of the at least one negative symptom associated with an autism spectrum disorder.

4. The method of claim 1, wherein the said usage data for a mobile device comprises data selected from the group consisting of: phone usage data, application (App) usage data, ambient noise data, movement capture data and location capture data.

5. The method of claim 1, wherein said at least one usage behavior parameter is a recorded variable selected from the list consisting of:
(i) phone and/or app usage;
(ii) ambient sound;
(iii) movement;
(iv) light and proximity data;
(v) touch behavior.

6. The method of claim 5, wherein an improvement of at least one negative symptom associated with an autism spectrum disorder is determined by improvements in the (i) phone and/or app usage, (ii) ambient sound, (iii) movement, (iv) light and proximity data and/or (v) touch behavior:
(i) wherein improvement in phone and/or app usage comprises increased number of contacts called, increased phone call duration and increased number of characters in SMS, decreased time and frequency of non-social apps and/or games, increase in frequency and time spent in social apps, decrease of the total amount of time spent using Apps, decreased unlock duration every time the patient uses the phone, increased number of networks (WIFI) and devices (bluetooth) during the day, decrease of duration connected to the most used network (home), and/or increased duration connected to networks different from the most used network;
(ii) wherein improvement in ambient sound comprises increased volume during the day, larger increases of ambient sound during the morning, higher pitch in voiced frames, increased ratio of voiced to non-voiced frames, increased duration in the voiced frames, and/or more time spent in social places;
(iii) wherein improvement in movement comprises increased activity during the day, decreased activity during the night, increased walk duration, longer walks, decreased duration of not moving, increased time on car travels, increased number of new places visited, longer distances covered during the day, and/or reduced time spent in a single place;
(iv) wherein improvement in light and proximity data comprises increased duration of the phone in the pocket, and/or decreased duration of use of the phone in the darkness; and
(v) wherein improvement in touch behavior comprises decreased activity and interaction in non-social apps and/or games, increased interaction with social apps; less browsing behavior in Apps, as measured by swipe gestures; changes to the circadian rhythm; increased amounts of typing behavior; increased amounts of typing behavior in social apps; increased use of certain punctuation marks; faster typing behavior.

7. The method of claim 1, wherein said reference is at least one usage behavior parameter which has been determined in a dataset comprising usage data for a mobile device within a second predefined time window prior to the first predefined time window.

8. The method of claim 7, wherein between the second and the first time windows the subject has received an autism spectrum disorder therapy or a therapy for at least of the negative symptoms associated therewith.

9. The method of claim 8, wherein said therapy is a drug-based therapy.

10. The method of claim 8, wherein an improvement of at least one negative symptom associated with an autism spectrum disorder is indicative for a successful therapy.

11. The method of claim 1, wherein said behavior data comprise one or more data selected from the group consisting of:
(i) data indicative for conversational skills and obsessive interest;
(ii) data indicative for sociability and routines;
(iii) data indicative for repetitive movements;
(iv) data indicative for sleep behavior;
(v) data indicative for anxiety;
(vi) data indicative for emotion recognition;
(vii) data indicative for spatial working memory;
(viii) data indicative for cooperation behavior; and
(ix) data indicative for image exploration capabilities, vocal properties and speaker recognition.

12. The method of claim 1, wherein said assessing ASD comprises assessing data indicative for at least one symptom associated with an autism spectrum disorder selected from the group consisting of: conversational skills and obsessive interests; repetitive movements; sleep behavior; anxiety; emotion recognition; spatial working memory; cooperation behavior; and image exploration capabilities, vocal properties and speaker recognition.

13. The method claim 12, wherein:
said data indicative for conversational skills and obsessive interest comprise data for voice characteristics, amount of speech and/or turn-taking behavior during conversations;
said data indicative for repetitive movements comprise data for frequency and duration of repetitive and/or stereotype movements;
said data indicative for sleep behavior comprise data for sleep latency, sleep efficiency, sleep time, waking after sleep onset and/or sleepiness;
said data indicative for anxiety comprise data for heart rate variability;
said data indicative for emotion recognition comprise data from a computer-implemented reading the mind in the eyes test (RMET);
said data indicative for spatial working memory comprise data from a computer-implemented test for working memory;
said data indicative for cooperation behavior comprise data from a computer-implemented test assessing cooperation behavior; and/or
said data indicative for image exploration capabilities, vocal properties and speaker recognition comprise data from a computer-implemented test for visually identifying social and non-social elements, voice characteristics, and/or speaker recognition by conversation and ambient sound.

14. The method of claim 1, wherein said mobile device is a smartphone, smartwatch, wearable sensor, portable multimedia device or tablet computer.

15. The method of claim 1, wherein said subject is a human.

16. A mobile device, comprising:
at least one sensor configured for recording usage data;
a database; and
a processor having stored thereon computer-executable instructions for performing the method according to claim 1.

17. A system comprising the mobile device as recited in claim 16 and a remote device operatively linked to the mobile device.

18. A method for recommending a therapy for ASD, comprising:
(a) assessing ASD by carrying out the method of claim 1; and
(b) recommending a therapy for ASD based on the assessment provided in step (a), wherein said therapy for ASD comprises treatment by at least one drug selected from the group consisting of: a Vasopressin 1a antagonist, a N-Methyl-D-Aspartate (NMDA) receptor antagonists, a selective inhibitor of the enzyme fatty acid amide hydrolase (FAAH), a GABA-modulator, a GABA A modulator or a selective GABA-B agonist, a mGlu4/7 positive allosteric modulator, oxytocin, an Acetyl-Choline Esterase Inhibitor, a dual inhibitor of lysine (K)-specific demethylase 1A/monoamine oxidase B, a tyrosine hydroxylase inhibitor, a selective and irreversible small molecule non-ATP-competitive glycogen synthase kinase 3 (GSK-3) inhibitor, an Amylase, lipase and protease regulator enzymes, a NKCC1 cation-chloride co-transporter blocker, a microbiota transfer therapy, a microbiome modulator, a selective serotonin reuptake inhibitor, a dopamine 2 receptor antagonist, a non-euphoric cannabinoid, a phytocannabinoid, a mu-opioid receptor antagonist.

19. A method of assessing ASD in a subject, comprising:
a) collecting the subject's usage data for a mobile device over a first predefined time window;
b) determining a usage behavior parameter from the usage data;
c) comparing the determined usage behavior parameter to a reference; and
d) determining an improvement, persistency or worsening of negative symptoms associated with ASD in the subject based on the comparison of step (c).

20. The method of claim 19, wherein said reference is a usage behavior parameter which has been determined from usage data from a mobile device within a second predefined time window prior to the first predefined time window.

21. The method of claim 19, comprising administering a therapy for ASD between the second and the first time windows.

22. The method of claim 21, wherein said therapy is a drug-based therapy.

23. The method of claim 22, wherein the drug-based therapy comprises treatment by at least one drug selected from the group consisting of: a Vasopressin 1a antagonist, a N-Methyl-D-Aspartate (NMDA) receptor antagonist, a selective inhibitor of the enzyme fatty acid amide hydrolase (FAAH), a GABA-modulator, a GABA A modulator or a selective GABA-B agonist, a mGlu4/7 positive allosteric modulator, oxytocin, a Acetyl-Choline Esterase Inhibitor, a dual inhibitor of lysine (K)-specific demethylase 1A/monoamine oxidase B, a tyrosine hydroxylase inhibitor, a selective and irreversible small molecule non-ATP-competitive glycogen synthase kinase 3 (GSK-3) inhibitor, Amylase, lipase and protease regulator enzymes, a NKCC1 cation-chloride co-transporter blocker, a microbiota transfer therapy, a microbiome modulator, a selective serotonin reuptake inhibitor, a dopamine 2 receptor antagonist, a non-euphoric cannabinoid, a phytocannabinoid, a mu-opioid receptor antagonist.
